(19) United States
(12) Patent Application Publication
(10) Pub. No.: US 2008/0274069 A1
(43) Pub. Date: Nov. 6, 2008

(54) Cosmetic Preparations Containing Copolymers of Ethyl Methacrylate and at Least One Monoethylenically Unsaturated Carboxylic Acid

(75) Inventors: Gabi Winter, Ludwigshafen (DE); Matthias Laubender, Schifferstadt (DE); Claudia Wood, Weinheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/577,672

(22) PCT Filed: Oct. 19, 2005
(86) PCT No.: PCT/EP05/11240

§ 371 Date: Apr. 20, 2007

(30) Foreign Application Priority Data
Oct. 22, 2004 (DE) 10 2004 051 647.2

(51) Int. Cl. A61K 8/81; C08F 226/10; A61Q 5/06; A61Q 5/04; A61Q 5/00

(52) U.S. Cl. 424/70.2; 526/258; 424/70.15

(57) ABSTRACT

Copolymers prepared by a process comprising free-radical polymerizing: (a) more than 15 to 40% by weight of at least one monoethylenically unsaturated carboxylic acid; (b) 30 to less than 85% by weight of ethyl methacrylate; and (c) 0 to 50% by weight of at least one additional free-radically polymerizable monomer other than the at least one monoethylenically unsaturated carboxylic acid and ethyl methacrylate; wherein the total amount of (a), (b) and (c) equals 100% by weight; with the proviso that where the process comprises a solution polymerization, (a) consists of acrylic acid and methacrylic acid, and (c) is an ester of (meth)acrylic acid and an aliphatic C₈-C₁₈-alcohol, then the ester of (meth)acrylic acid and an aliphatic C₈-C₁₈-alcohol is not present in an amount of 5 to 40% by weight; and with the proviso that where (c) is selected from the group consisting of N-vinylpyrrolidone, N-vinylcaprolactam and mixtures thereof; then the N-vinylpyrrolidone, N-vinylcaprolactam or mixture thereof is present in an amount of less than 5% by weight.

The present invention relates to cosmetic and pharmaceutical compositions comprising copolymers which comprise ethyl methacrylate and at least one monoethylenically unsaturated carboxylic acid in copolymerized form, to the copolymers themselves, and to the use thereof.

### PRIOR ART

Polymers with film-forming properties have found diverse applications in the field of pharmacy and cosmetics. In pharmacy, for example, they serve as coating compositions or binders for solid drug forms. In cosmetics, polymers with film-forming properties are used, inter alia, for setting, improving the structure and shaping of hair. They serve, for example, as conditioners to improve the dry and wet combability, the feel to the touch, the shine and the appearance, and to impart antistatic properties to the hair. Requirements which are placed on film-forming polymers for use as setting resins are, for example, strong hold (even at high atmospheric humidity), elasticity, ability to be washed out of the hair, compatibility in the formulation and a pleasant feel of the hair treated therewith. The preparation of products with a complex profile of properties often presents problems. There is thus a need for film-forming polymers for hair cosmetic compositions which are able to form essentially smooth, tack-free films, have a good setting action and at the same time impart good sensorily ascertainable properties, such as elasticity and a pleasant feel, to the hair. If these polymers are to be used in hair spray formulations, then good propellant gas compatibility, suitability for use in low-VOC formulations (VOC=Volatile Organic Compounds), good sprayability, good solubility in water or aqueous/alcoholic solvent mixtures and good ability to be washed out is also desired.

Copolymers based on (meth)acrylate which are water-soluble in an alkaline medium are often used in the field of cosmetics as hair-setting agents.

Thus, for example EP-A-379 082 describes hair-setting compositions which comprise, as film formers, copolymers with a K value of from 10 to 50 which are constructed from 75 to 99% by weight of t-butyl acrylate and/or t-butyl methacrylate, 1 to 25% by weight of acrylic acid and/or methacrylic acid and 0 to 10% by weight of a further free-radically copolymerizable monomer. For use in cosmetics, the carboxyl groups of the copolymers are partially or completely neutralized by amines.

DE-A-43 14 305 describes hair-setting compositions which comprise, as film formers, copolymers of the type specified in EP-A-379 082 and constructed from 30 to 72% by weight of t-butyl acrylate and/or t-butyl methacrylate, 10 to 28% by weight of acrylic acid and/or methacrylic acid and 0 to 60% by weight of a free-radically copolymerizable monomer or a free-radically copolymerizable monomeric mixture, where at least one of these monomers produces a homopolymer with a glass transition temperature below 30° C.

U.S. Pat. No. 3,577,517 describes hair lacquers and aerosol preparations which comprise neutralized solution polymers of 5 to 40% by weight of a (meth)acrylic ester with aliphatic C₈-C₁₈-alcohols, 6 to 35% by weight of (meth)acrylic acid and 25 to 89% by weight of a further vinyl monomer, a water-soluble organic solvent, and propellant. Polymers obtainable by emulsion polymerization are not described.

U.S. Pat. No. 5,589,157 and EP-A 590 604 describe aqueous acrylic polymer compositions which comprise copolymers which are constructed from 35 to 74% by weight of an alkyl acrylate, 25 to 65% by weight of an alkyl methacrylate and 1 to 15% by weight of a (meth)acrylic acid or salts thereof, and their use in hair cosmetic preparations.

JP 3056409 describes hair spray preparations comprising an anionic resin which is constructed from 30 to 70% by weight of C₁-C₇-alkyl or -alkenyl(meth)acrylate, 10 to 50% by weight of C₉-C₂₄-alkyl or -alkenyl(meth)acrylate and 10 to 40% by weight of a polymerizable carboxyl-containing compound.

U.S. Pat. No. 3,405,084 describes terpolymer resins consisting of 25 to 75% by weight of vinyl-pyrrolidone, 20 to 70% by weight of (meth)acrylate and 3 to 25% by weight of olefinically unsaturated carboxylic acid for use in hair cosmetic preparations, in particular as hair sprays.

EP-A 331 994 describes hair-setting preparations comprising copolymers of a) 40-60% by weight of C₃-C₁₂-alkyl methacrylates, b) 20 to 40% by weight of C₄-C₁₀-N-alkyl-substituted acrylamides and c) 10 to 25% by weight of (meth)acrylic acid. Preference is given to using copolymers of a) isobutyl methacrylate, b) N-tert-octylacrylamide and c) acrylic acid.

EP-A 985 401 describes hair styling preparations comprising a resin consisting of 5-95% C₁-C₁₀-alkyl(meth)acrylate, 2-16% hydroxyalkyl(meth)acrylate, 0-50% C₃-C₈ monoethylenically unsaturated monocarboxylic acids and 2-10% monoethylenically unsaturated dicarboxylic acids.

GB 1410012 describes aerosol hair spray preparations which comprise methacrylic acid-C₁-C₃-alkyl(meth)acrylate copolymers, in particular methyl acrylate copolymers or methacrylic acid-methyl acrylate-methyl methacrylate terpolymers.

EP-A 62 002 describes hair cosmetic preparations comprising terpolymers which are prepared by copolymerization of a) 40 to 60% by weight of a N-alkylacrylamide or N-alkylmethacrylamide having 1 to 4 carbon atoms in the alkyl moiety with b) 35 to 50% by weight of a C₁-C₄-hydroxyalkyl ester or preferably C₁-C₄-alkyl ester of acrylic acid or methacrylic acid and c) 3 to 11% by weight of an α,β-unsaturated monocarboxylic acid or dicarboxylic acid.

DE 42 23 006 describes hair-treatment compositions which comprise, as film formers, copolymers which are obtainable by copolymerization of (a) 30 to 80% by weight of an acrylic or methacrylic ester which, in each case as homopolymer, has a glass transition temperature of more than 20° C. or of mixtures of acrylic and methacrylic esters which produce copolymers with a glass transition temperature of more than 20° C. during the copolymerization, (b) 5 to 25% by weight of acrylic acid, methacrylic acid or mixtures thereof and (c) 10 to 45% by weight of N-vinylpyrrolidone, N-vinylcaprolactam or mixtures thereof in the presence of free-radical-forming polymerization initiators, and which, in the form of the free carboxylic acid groups, have K values of (determined in accordance with H. Fikentscher in 1% strength by weight solution in ethanol at 25° C.) of from 10 to 80, wherein the copolymers are prepared by the method of precipitation polymerization.

EP-A 100 890 discloses copolymers obtained by free-radical copolymerization of
a) 20 to 75 parts by weight of at least one C₂-C₂₀-alkyl ester of (meth)acrylic acid,
b) 5 to 50 parts by weight of at least one nitrogen-containing, neutrally reacting water-soluble monomer,
c) 1 to 25 parts by weight of at least one monomer comprising cationic groups and
d) 1 to 25 parts by weight of at least one olefinically unsaturated C₃-C₈-carboxylic acid copolymerizable with a), b) and c), and which, measured in ethanol at 25° C., have a K value according to Fikentscher of from 15 to 75.

More stringent environmental conditions and increasing ecological awareness increasingly demand ever lower fractions of volatile organic components (VOCs) in, for example, hair sprays.

The VOC content in hair sprays is essentially set by the nonaqueous solvents and the propellants. For this reason, instead of nonaqueous solvents, recourse has increasingly been made to water as solvent. However, this replacement of the organic solvents brings with it a number of problems, particularly in the field of hair spray formulations.

Thus, formulations of the abovementioned film-forming polymers from the prior art which satisfy the corresponding VOC conditions are, for example, not sprayable or sprayable only after further dilution and are thus only of limited suitability for use in hair sprays. This in turn leads to films which sometimes do not have the required mechanical quality and thus inadequate setting effect and poor hold for the hair.

Requirements placed on hair-setting resins are, for example, strong hold at high atmospheric humidity, elasticity, good ability to be washed out of the hair, compatibility in the formulation, lowest possible tack of the film formed and a pleasant feel of the hair treated therewith. For spray formulations, a homogeneous distribution of small droplets for forming a fine spray pattern is in particular also desired. The preparation of products with a complex profile of properties often presents difficulties. There is thus a need for particularly cosmetic preparations which are able to form essentially smooth, tack-free films which impart good sensorily ascertainable properties to the hair and to the skin, such as a pleasant feel, and at the same time have a good conditioning effect and/or setting effect.

The object of the present invention was to provide polymers for cosmetic preparations which can be formulated in solvents or solvent mixtures with an increased water content and whose formulations, upon use in the form of a spray, have good sprayability and a fine homogeneous spray pattern coupled with good mechanical properties of the films formed. Besides the good compatibility with the customary cosmetic ingredients, the polymers should impart good setting and longer hold to the hair, have good ability to be washed out and be able to be formulated as optically clear VOC 55 aerosols (i.e. with a VOC content of at most 55% by weight).

Surprisingly, it has been found that the ethyl methacrylate copolymers according to the invention achieve this object.

In that which follows compounds which can be derived from acrylic acid and methacrylic acid are sometimes referred to in short by adding the syllable “(meth)” to the compound derived from acrylic acid.

The invention provides cosmetic preparations comprising at least one copolymer obtainable by free-radical polymerization of
a) more than 15 to 40% by weight of at least one monoethylenically unsaturated carboxylic acid,
40 b) 30 to less than 85% by weight of ethyl methacrylate,
c) 0 to 50% by weight of at least one further free-radically polymerizable monomer
c) different from a) and b), where the amounts of components a), b) and c) add up to 100% by weight,
   with the proviso that
   if the free-radical polymerization is a solution polymerization and a) consists exclusively of acrylic acid and methacrylic acid and at the same time c) is chosen from the group of esters of (meth)acrylic acid with aliphatic C₈-C₁₈-alcohols, c) must then not be chosen from 5 to 40% by weight of esters of (meth)acrylic acid with aliphatic C₈-C₁₈-alcohols and
   with the proviso that
   if c) is chosen from the group consisting of N-vinylpyrrolidone, N-vinylcaprolactam and mixtures thereof, a) is then present in an amount of less than 10% by weight.

Accordingly, the invention also provides polymers obtainable by free-radical polymerization of
a) 16 to 40% by weight of at least one monoethylenically unsaturated carboxylic acid,
b) 30 to less than 84% by weight of ethyl methacrylate,
c) 0 to 50% by weight of at least one further free-radically polymerizable monomer
c) different from a) and b), where the amounts of components a), b) and c) add up to 100% by weight,
   with the proviso that
   if the free-radical polymerization is a solution polymerization and a) consists exclusively of acrylic acid and methacrylic acid and at the same time c) is chosen from the group of esters of (meth)acrylic acid with aliphatic C₈-C₁₈-alcohols, c) must then not be chosen from 5 to 40% by weight of esters of (meth)acrylic acid with aliphatic C₈-C₁₈-alcohols and
   with the proviso that
   if c) is chosen from the group consisting of N-vinylpyrrolidone, N-vinylcaprolactam and mixtures thereof c) is then present in an amount of less than 10% by weight.

Accordingly, the invention also provides polymers obtainable by free-radical polymerization of a) 16 to 40% by weight of at least one monoethylenically unsaturated carboxylic acid, b) 30 to 84% by weight of ethyl methacrylate, c) 0 to 50% by weight of at least one further free-radically polymerizable monomer c) different from a) and b), where the amounts of components a), b) and c) add up to 100% by weight, with the proviso that if the free-radical polymerization is a solution polymerization and a) consists exclusively of acrylic acid and methacrylic acid and at the same time c) is chosen from the group of esters of (meth)acrylic acid with aliphatic C₈-C₁₈-alcohols, a) must then not be chosen from 5 to 40% by weight of esters of (meth)acrylic acid with aliphatic C₈-C₁₈-alcohols and with the proviso that if c) is chosen from the group consisting of N-vinylpyrrolidone, N-vinylcaprolactam and mixtures thereof, c) is then present in an amount of less than 10% by weight.

The object is also achieved by the provision of cosmetic preparations comprising at least one copolymer obtainable by free-radical polymerization of
a) 16 to 40% by weight of at least one monoethylenically unsaturated carboxylic acid,
b) 30 to 84% by weight of ethyl methacrylate,
c) 0 to 50% by weight of at least one further free-radically polymerizable monomer c) different from a) and b), where the amounts of components a), b) and c) add up to 100% by weight, and where the polymerization is an emulsion polymerization.

Accordingly, the invention also provides polymers obtainable by free-radical polymerization of a) 16 to 40% by weight of at least one monoethylenically unsaturated carboxylic acid, b) 30 to 84% by weight of ethyl methacrylate, c) 0 to 50% by weight of at least one further free-radically polymerizable monomer c) different from a) and b), where the amounts of components a), b) and c) add up to 100% by weight, and where the polymerization is an emulsion polymerization.

The invention further provides polymers obtainable by free-radical polymerization of a) 5 to 40% by weight, preferably 10 to 35% by weight, particularly preferably 12 to 30% by weight, of at least one monoethylenically unsaturated carboxylic acid, b) 30 to 95% by weight, preferably 40 to 80% by weight, particularly preferably 45 to 75% by weight, of ethyl methacrylate, c) 0 to 50% by weight of at least one further free-radically polymerizable monomer c) different from a) and b), where the amounts of a), b) and c) add up to 100% by weight

with the proviso
that if the free-radical polymerization is a solution polymerization and a) consists exclusively of acrylic acid and methacrylic acid and at the same time c) is chosen from the group of esters of (meth)acrylic acid with aliphatic C₈-C₁₈-alcohols, c) must then not be chosen from 5 to 40% by weight of esters of (meth)acrylic acid with aliphatic C₈-C₁₈-alcohols and
with the proviso that
if c) is chosen from the group consisting of N-vinylpyrrolidone, N-vinylcaprolactam and mixtures thereof c) is then present in an amount of less than 10% by weight and with the proviso that less than 35% by weight of the monomers are chosen from C₁-C₅-alkyl acrylate.

### Component a)

The monomers a) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers a) also include the half-esters of monoethylenically unsaturated dicarboxylic acids having 4 to 10, preferably 4 to 6, carbon atoms, e.g. of maleic acid, such as monomethyl maleate. The monomers a) also include the salts of the above-mentioned acids, in particular the sodium, potassium and ammonium salts. The monomers a) can be used as such or as mixtures with one another. All of the weight fractions given refer to the acid form.

Preferably, monomer a) is chosen from the group consisting of acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, particularly preferably acrylic acid, methacrylic acid and mixtures thereof and in particular methacrylic acid.

In a preferred embodiment of the invention, a) consists of a1) methacrylic acid and if appropriate a2) further monoethylenically unsaturated monocarboxylic acids different from methacrylic acid.

In a preferred embodiment of the invention a) consists of a1) methacrylic acid and optionally a2), where a2) is chosen from the group consisting of acrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof.

In a further preferred embodiment of the invention, a) consists of a1) acrylic acid and optionally a2), where a2) is chosen from the group consisting of methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof.

In a further preferred embodiment, component a) comprises no monoethylenically unsaturated dicarboxylic acids.

In a further preferred embodiment, component a) comprises a mixture of methacrylic acid and itaconic acid or component a) consists of a mixture of methacrylic acid and itaconic acid.

In a further preferred embodiment, component a) comprises a mixture of methacrylic acid and acrylic acid or component a) consists of a mixture of methacrylic acid and acrylic acid.

If component a) is a mixture of methacrylic acid and further monoethylenically unsaturated carboxylic acids, it is advantageous if the weight ratio of methacrylic acid to further acids is greater than 1, preferably greater than 2, particularly preferably at least 3 and especially at least 4.

According to the invention, the amount of a) is at least 16, preferably at least 18, particularly preferably at least 20 and at most 40, preferably at most 38, particularly preferably at most 35 and very particularly preferably at most 30% by weight.

In a preferred embodiment, the polymers according to the invention consist exclusively of ethyl methacrylate and component a).

### Component c)

To prepare the polymers according to the invention, if appropriate 0 to 50% by weight of a monomer c) are used.

Esters of α,β-ethylenically Unsaturated mono- and dicarboxylic Acids

Suitable monomers c) are, for example, chosen from C₁-C₁₈-alkyl acrylates and C₁-C₁₈-alkyl methacrylates.

Suitable monomers c) are the esters of α,β-ethylenically unsaturated mono- and dicarboxylic acids different from ethyl methacrylate, such as, for example, methyl(meth)acrylate, ethyl acrylate, methyl ethacrylate, n-propyl(meth)acrylate, isopropyl(meth)acrylate, n-propyl ethacrylate, ethyl ethacrylate, isopropyl ethacrylate, n-butyl(meth)acrylate, n-butyl ethacrylate, tert-butyl(meth)acrylate, tert-butyl ethacrylate, iso-butyl(meth)acrylate, isobutyl ethacrylate, sec-butyl(meth)acrylate, butyl ethacrylate, 2-pentyl(meth)acrylate, 3-pentyl(meth)acrylate, isopentyl acrylate, neopentyl acrylate, hexyl(meth)acrylate, 1-methyl pentyl(meth)acrylate, 2-methylpentyl(meth)acrylate, 3-methylpentyl(meth)acrylate, 4-methylpentyl(meth)acrylate, 1,1-dimethylbutyl(meth)acrylate, 1,2-dimethylbutyl(meth)acrylate, 1,3-dimethylbutyl(meth)acrylate, 2,2-dimethylbutyl(meth)acrylate, 2,3-dimethylbutyl(meth)acrylate, 3,3-dimethylbutyl(meth)acrylate, 1-ethylbutyl(meth)acrylate, 1,1,2-trimethylpropyl(meth)acrylate, 1,2,2-trimethylpropyl-(meth)acrylate, 1-ethyl-1-methylpropyl(meth)acrylate, 1-ethyl-2-methylpropyl, n-heptyl(meth)acrylate, n-octyl(meth)acrylate, 1,13,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl (meth)acrylate, pentadecyl(meth)acrylate, palmityl(meth)acrylate, heptadecyl (meth)acrylate, nonadecyl(meth)acrylate, arrachidyl(meth)acrylate, behenyl (meth)acrylate, lignocerenyl(meth)acrylate, cerotinyl(meth)acrylate, melissinyl (meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate, phenoxyethyl acrylate, t-butylcyclohexyl acrylate, cyclohexyl(meth)acrylate, ureido(meth)acrylate, tetrahydrofurfuryl(meth)acrylate, ureido methacrylate and mixtures thereof.

Preferred monomers c) are the esters of α,β-ethylenically unsaturated mono- and dicarboxylic acids with C₁-C₄-alkanols different from ethyl methacrylate.

### Hydroxyalkyl(meth)acrylates

Suitable monomers c) are also esters of α,β-ethylenically unsaturated mono- and dicarboxylic acids with polyhydric alcohols, such as, for example, 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate, 3-hydroxy-2-ethylhexyl methacrylate and mixtures thereof.

If a) consists exclusively of acrylic acid and methacrylic acid and at the same time component c) is chosen from the group of esters of (meth)acrylic acid with aliphatic C₈-C₁₈-alcohols, then c) is not chosen from 5 to 40% by weight of esters of (meth)acrylic acid with aliphatic C₈-C₁₈-alcohols.

Suitable monomers c) are also vinyl esters, such as, for example, vinyl acetate, vinyl propionate, vinyl butyrate, vinyl ester of versatic acid (VeoVa® monomers), vinyl 4-tert-butylbenzoate and mixtures thereof.

Suitable monomers c) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

N-vinylamides of Saturated C₁-C₈-monocarboxylic Acids

Suitable monomers c) are also N-vinylamides of saturated C₁-C₈-monocarboxylic acids, such as N-vinylformamide, N-vinyl-N-methylformamide, N-vinylacetamide, N-vinyl-N-methylacetamide, N-vinyl-N-ethylacetamide, N-vinylpropionamide, N-vinyl-N-methyl-propionamide, N-vinylbutyramide and mixtures thereof.

Amides of α,β-ethylenically Unsaturated mono- and dicarboxylic Acids

Suitable monomers c) are also N—C₁-C₁₈-alkylacrylamides and N—C₁-C₁₈-alkyl-methacrylamides.

Suitable monomers c) are also in particular primary amides of α,β-ethylenically unsaturated monocarboxylic acids and their N-alkyl and N,N-dialkyl derivatives which, in addition to the carbonyl carbon atom of the amide group, have at most 8 further carbon atoms, such as, for example, acrylamide, methacrylamide, N-methyl(meth)acrylamide, N-ethyl(meth)acrylamide, N-propyl(meth)acrylamide, N-(n-butyl)methacrylamide, N-(sec-butyl)methacrylamide, N-(tert-butyl)methacrylamide, N-(n-pentyl)(meth)acrylamide, N-(n-hexyl)(meth)acrylamide, N-(n-heptyl)(meth)-acrylamide, N-(n-octyl)(meth)acrylamide, N-(tert-octyl)(meth)acrylamide, N-(1,1,3,3-tetramethylbyutyl)(meth)acrylamide, N-ethylhexyl(meth)acrylamide, N-(n-nonyl)(meth)-acrylamide, N-(n-decyl)(meth)acrylamide, N-(n-undecyl)(meth)acrylamide, N-tridecyl-(meth)acrylamide, N-myristyl(meth)acrylamide, N-pentadecyl(meth)acrylamide, N-palmityl(meth)acrylamide, N-heptadecyl(meth)acrylamide, N-nonadecyl-(meth)acrylamide, N-arrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide, N,N-dimethyl(meth)acrylamide, N,N-diethyl(meth)acrylamide piperidinyl(meth)acrylamide and morpholinyl(meth)acrylamide and mixtures thereof.

In a preferred embodiment of the invention, c) comprises methacrylamide and/or N-(tert-butyl)acrylamide or consists of one of these components or of a mixture of methacrylamide and N-(tert-butyl)acrylamide.

Suitable monomers c) are also amides of α,β-ethylenically unsaturated mono- and dicarboxylic acids with aminoalcohols which have a primary or secondary amino group, such as 2-hydroxyethylacrylamide, 2-hydroxyethylmethacrylamide, 2-hydroxy-ethylethacrylamide, 2-hydroxypropylacrylamide, 2-hydroxypropylmethacrylamide, 3-hydroxypropylacrylamide, 3-hydroxypropylmethacrylamide, 3-hydroxybutyl-acrylamide, 3-hydroxybutylmethacrylamide, 4-hydroxybutylacrylamide, 4-hydroxybutylmethacrylamide, 6-hydroxyhexylacrylamide, 6-hydroxyhexyl-methacrylamide, 3-hydroxy-2-ethylhexylacrylamide, 3-hydroxy-2-ethylhexylmethacrylamide and mixtures thereof.

The copolymers according to the invention can additionally comprise at least one compound with a free-radically polymerizable α,β-ethylenically unsaturated double bond and at least one cationogenic and/or cationic group per molecule in copolymerized form.

The cationogenic and/or cationic groups of component c) are preferably nitrogen-containing groups, such as primary, secondary and tertiary amino groups, and quaternary ammonium groups. The nitrogen-containing groups are preferably tertiary amino groups. Preference is given to using the compounds c) in non-charged form for the polymerization. However, a use in charged form is also possible. Charged cationic groups can be produced, for example, from the amine nitrogens by protonation, e.g. with monobasic or polybasic carboxylic acids, such as lactic acid or tartaric acid, or mineral acids, such as phosphoric acid, sulfuric acid and hydrochloric acid.

Component c) can be chosen from esters of α,β-ethylenically unsaturated mono- and dicarboxylic acids with aminoalcohols which may be mono- or dialkylated on the amine nitrogen, amides of α,β-ethylenically unsaturated mono- and dicarboxylic acids with diamines which have at least one primary or secondary amino group, N,N-dialkylamine, N,N-diallyl-N-alkylamines and derivatives thereof vinyl- and allyl-substituted nitrogen heterocycles, vinyl- and allyl-substituted heteroaromatic compounds and mixtures thereof.

Suitable as acid component of these esters are, for example, acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof. Examples of such compounds c) are N-methylaminoethyl(meth)acrylate, N-ethylaminoethyl(meth)acrylate, N-(n-propyl)aminoethyl(meth)acrylate, N-(n-butyl)aminoethyl(meth)acrylate, N-(tert-butyl)aminoethyl(meth)acrylate, N,N-dimethylaminomethyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate and N,N-dimethylaminocyclohexyl(meth)acrylate.

In particular, N-(tert-butyl)aminoethyl acrylate and N-(tert-butyl)aminoethyl methacrylate are used as compound c).

Suitable monomers c) are also the amides of the abovementioned α,β-ethylenically unsaturated mono- and dicarboxylic acids with diamines which have at least one primary or secondary amino group. Preference is given to diamines which have one tertiary and one primary or secondary amino group. As monomers c), preference is given to using N-[2-(dimethylamino)ethyl]acrylamide,
N-[2-(dimethylamino)ethyl]methacrylamide, N-[3-(dimethylamino)propyl]acrylamide,
N-[3-(dimethylamino)propyl]methacrylamide, N-[4-(dimethylamino)butyl]acrylamide,
N-[4-(dimethylamino)butyl]methacrylamide, N-[2-(diethylamino)ethyl]acrylamide,
N-[4-(dimethylamino)cyclohexyl]acrylamide and N-[4-(dimethylamino)cyclohexyl]-methacrylamide. Particular preference is given to using N-[3-(dimethylamino)propyl]-acrylamide and/or N-[3-(dimethylamino)propyl]methacrylamide.

These cationogenic monomers c) can also be present in quaternized form. Quaternization takes place in the case of the abovementioned monomers if appropriate using methyl chloride, methyl sulfate or diethyl sulfate.

Suitable monomers c) are also N,N-dialkylamines and N,N-diallyl-N-alkylamines and acid addition salts thereof. Alkyl here is preferably C₁-C₂₄-alkyl. Preference is given, for example, to N,N-diallyl-N-methylamine.

These cationogenic monomers c) can also be present in quaternized form. Quaternization takes place with customary quaternizing agents such as, for example, methyl chloride, methyl sulfate or diethyl sulfate.

Suitable monomers c) are also vinyl- and allyl-substituted nitrogen heterocycles, such as N-vinylimidazole, N-vinylimidazole derivatives, e.g. N-vinyl-2-methylimidazole, vinyl- and allyl-substituted heteroaromatic compounds, such as 2- and 4-vinylpyridine, 2- and 4-allylpyridine, and the salts thereof.

Suitable monomers c) are also N-vinylimidazoles of the general formula (I) in which R¹ to R³ is hydrogen, C₁-C₄-alkyl or phenyl

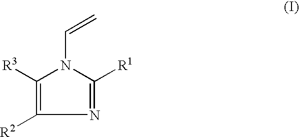

Examples of compounds of the general formula (I) are given in Table 1 below:

TABLE 1

| R¹ | R² | R³ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

In a preferred embodiment of the invention, component c) is chosen from N-(tert-butylamino)ethyl (meth)acrylate, N,N-dimethylaminoethyl(meth)acrylate, N-[3-(dimethylamino)propyl] (meth)acrylamide, vinylimidazole and mixtures thereof.

These cationogenic monomers c) can also be present in quaternized form. Quaternization takes place with customary quaternizing agents such as, for example, methyl chloride, methyl sulfate or diethyl sulfate.

### Polyether Acrylates

Suitable components c) are also polyether acrylates. For the purposes of this invention, polyether acrylates are generally understood as meaning esters of α,β-ethylenically unsaturated mono- and dicarboxylic acids with polyetherols. Suitable polyetherols are linear or branched substances having terminal hydroxyl groups which comprise ether bonds. In general, they have a molecular weight in the range from about 150 to 20 000. Suitable polyetherols are polyalkylene glycols, such as polyethylene glycols, polypropylene glycols, polytetrahydrofurans and alkylene oxide copolymers. Suitable alkylene oxides for preparing alkylene oxide copolymers are, for example, ethylene oxide, propylene oxide, epichlorohydrin, 1,2- and 2,3-butylene oxide. The alkylene oxide copolymers can comprise the copolymerized alkylene oxide units in random distribution or in the form of blocks. Preference is given to ethylene oxide/propylene oxide copolymers.

Preferred components c) are polyether acrylates of the general formula V

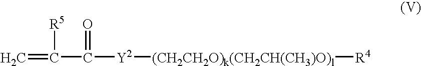

in which
the order of the alkylene oxide units is arbitrary,
k and l, independently of one another, are an integer from 0 to 1000, where the sum of k and l is at least 5,
R⁴ is hydrogen, C₁-C₃₀-alkyl or C₅-C₈-cycloalkyl,
R⁵ is hydrogen or C₁-C₈-alkyl,
Y² is O or NR⁶, where R⁶ is hydrogen, C₁-C₃₀-alkyl or C₅-C₈-cycloalkyl.

Preferably, k is an integer from 1 to 500, in particular 3 to 250. Preferably, l is an integer from 0 to 100.

Preferably, R⁵ is hydrogen, methyl, ethyl, n-propyl, isopropyl, n-butyl, sec-butyl, tert-butyl, n-pentyl or n-hexyl, in particular hydrogen, methyl or ethyl.

Preferably, R⁴ in the formula V is hydrogen, methyl, ethyl, n-propyl, isopropyl, n-butyl, sec-butyl, n-pentyl, n-hexyl, octyl, 2-ethylhexyl, decyl, lauryl, palmityl or stearyl.

Preferably, Y² in the formula V is O or NH.

Suitable polyether acrylates c) are, for example, the polycondensation products of the abovementioned α,β-ethylenically unsaturated mono- and/or dicarboxylic acids and their acid chlorides, amides and anhydrides with polyetherols. Suitable polyetherols can be prepared easily by reacting ethylene oxide, 1,2-propylene oxide and/or epichlorohydrin with a starter molecule, such as water or a short-chain alcohol R⁴—OH. The alkylene oxides can be used individually, alternately one after the other or as a mixture. The polyether acrylates c) can be used on their own or in a mixture for preparing the polymers used according to the invention.

Suitable components c) are also allyl alcohol propoxylates or allyl alcohol ethoxylates.

Suitable monomers c) are also N-vinyllactams, are unsubstituted N-vinyllactams and N-vinyllactam derivatives which have, for example, one or more C₁-C₆-alkyl substituents, such as methyl ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc.

The copolymers according to the invention preferably comprise less than 10% by weight, preferably less than 5% by weight, particularly preferably less than 3% by weight, of N-vinyllactams in copolymerized form. The copolymers according to the invention very particularly preferably comprise less than 1% by weight and in particular no N-vinyllactams in copolymerized form.

In cases where monomer c) is chosen from the group consisting of N-vinylpyrrolidone, N-vinylcaprolactam and mixtures thereof, the amount of c) is less than 10, preferably less than 5% by weight, particularly preferably less than 3 and especially less than 1% by weight.

In preferred cosmetic preparations, the at least one monomer c) is chosen from the group consisting of C₁-C₁₈-alkyl acrylates, C₁-C₁₈-alkyl methacrylates, acrylamide, methacrylamide, N—C₁-C₁₈-alkylacrylamides and N—C₁-C₁₈-alkylmethacrylamides, where n-butyl acrylate, tert-butyl acrylate, hydroxyethyl methacrylate, ethyl acrylate and mixtures thereof are particularly preferred.

The abovementioned monomers c) can in each case be used individually or in the form of any desired mixtures.

A preferred cosmetic formulation comprises a copolymer obtainable by free-radical polymerization of
a) 18 to 35% by weight of a monoethylenically unsaturated carboxylic acid,
b) 40 to 80% by weight of ethyl methacrylate,
c) 0 to 50% by weight of monomer c),
   where the amounts of components a), b) and c) add up to 100% by weight.

A particularly preferred cosmetic formulation comprises a copolymer obtainable by free-radical polymerization of
a) 20 to 35% by weight of a monoethylenically unsaturated carboxylic acid,
b) 45 to 80% by weight of ethyl methacrylate,
c) 0 to 50% by weight of monomer c),
   where the amounts of components a), b) and c) add up to 100% by weight.

The monomers c) are used in amounts of from 0 to 50% by weight, preferably in amounts of from 0 to 30, particularly preferably in amounts of from 0 to 20% by weight, very particularly preferably in amounts of from 0 to 15% by weight and especially in amounts of less than 10% by weight.

### Crosslinkers

The copolymers according to the invention can, if desired, comprise at least one crosslinker, i.e. a compound with two or more than two ethylenically unsaturated, nonconjugated double bonds, in copolymerized form.

Crosslinkers are preferably used in an amount of from 0.01 to 7% by weight, particularly preferably 0.1 to 5% by weight, based on the total weight of the monomers used for the polymerization.

Suitable crosslinkers are, for examples acrylic esters, methacrylic esters, allyl ethers or vinyl ethers of at least dihydric alcohols. The OH groups of the parent alcohols can here be completely or partially etherified or esterified; however, the crosslinkers comprise at least two ethylenically unsaturated groups.

Examples of the parent alcohols are dihydric alcohols such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of from in each case 200 to 10 000. Apart from the homopolymers of ethylene oxide and propylene oxide, it is also possible to use block copolymers of ethylene oxide or propylene oxide or copolymers which comprise ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates. The polyhydric alcohols can also firstly be converted to the corresponding glycidyl ethers by reaction with epichlorohydrin.

Further suitable crosslinkers are the vinyl esters or the esters of monohydric unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-otadecen-1-ol. It is, however, also possible to esterify the monohydric unsaturated alcohols with polybasic carboxylic acids, for example malonic acid, tartaric acid, trimellitic acid, phthalic acid, terephthalic acid, citric acid or succinic acid.

Further suitable crosslinkers are esters of unsaturated carboxylic acids with the above-described polyhydric alcohols, for example of oleic acid, crotonic acid, cinnamic acid or 10-undecenoic acid.

Suitable crosslinkers are also straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Suitable crosslinkers are also the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as has been described above.

Also suitable as crosslinkers are triallylamine and triallylmonoalkylammonium salts, e.g. triallylmethylammonium chloride or methyl sulfate.

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers are divinyldioxane, tetraallyisilane or tetravinylsilane. Mixtures of the abovementioned compounds can of course also be used. Preference is given to using water-soluble crosslinkers.

Crosslinkers used with particular preference are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred crosslinkers are pentaerythritol triallyl ether, methylenebis-acrylamide, N,N′-divinylethyleneurea, triallylamine and triallylmonoalkylammonium salts and acrylic esters of glycol, butanediol, trimethylolpropane or glycerol or acrylic esters of glycol, butanediol, trimethyloipropane or glycerol reacted with ethylene oxide and/or epichlorohydrin.

### Preparation of the Copolymers

The copolymers are prepared by customary methods known to the person skilled in the art, e.g. by solution, precipitation, suspension or emulsion polymerization.

Preference is given to preparation by solution or emulsion polymerization.

Preferred solvents for the solution polymerization are aqueous solvents such as water, and mixtures of water with water-miscible solvents, for example alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of dihydric alcohols, diethylene glycol, triethylene glycol, polyethylene glycols with number-average molecular weights up to about 3000, glycerol and dioxane.

Particular preference is given to polymerization in water or a water/alcohol mixture, for example in a water/ethanol mixture. When using water as solvent constituent, demineralized water is preferably used.

The copolymers are particularly preferably prepared in a known manner by free-radically initiated aqueous emulsion polymerization of the monomers a), b) and if appropriate c).

### Emulsion Polymerization

The method of free-radically initiated aqueous emulsion polymerization has already been described widely and is therefore sufficiently known to the person skilled in the art [cf. e.g. Encyclopedia of Polymer Science and Engineering, Vol. 8, pages 659 to 677, John Wiley & Sons, Inc., 1987; D. C. Blackley, Emulsion Polymerisation, pages 155 to 465, Applied Science Publishers, Ltd., Essex, 1975; D. C. Blackley, Polymer Latices, 2^{nd} Edition, Vol. 1, pages 33 to 415, Chapman & Hall, 1997; H. Warson, The Applications of Synthetic Resin Emulsions, pages 49 to 244, Ernest Benn, Ltd., London, 1972; D. Diederich, Chemie in unserer Zeit 1990, 24, pages 135 to 142, Verlag Chemie, Weinheim; J. Piirma, Emulsion Polymerisation, pages 1 to 287, Academic Press, 1982; F. Hölscher, Dispersionen synthetischer Hochpolymerer, pages 1 to 160, Springer-Verlag, Berlin, 1969 and DE-A 40 03 422]. The free-radically initiated aqueous emulsion polymerization is usually carried out such that the monomers, usually with co-use of dispersants, are dispersely distributed within the aqueous medium and polymerized by means of at least one free-radical polymerization initiator.

### Initiators

Suitable free-radical polymerization initiators for the free-radical aqueous emulsion polymerization according to the invention are all those which are able to trigger a free-radical aqueous emulsion polymerization.

In principle, these may either be peroxides or azo compounds. Redox initiator systems are of course also suitable.

Peroxides which may be used are, in principle, inorganic peroxides, such as hydrogen peroxide or peroxodisulfates, such as the mono- or di-alkali metal or ammonium salts of peroxodisulfuric acid, such as, for example, its mono- and di-sodium, -potassium or ammonium salts or organic peroxides, such as alkyl hydroperoxides, for example tert-butyl, p-menthyl or cumyl hydroperoxide, tert-butyl perpivalate, and dialkyl or diaryl peroxides, such as di-tert-butyl or di-cumyl peroxide, 2,5-dimethyl-2,5-di(t)butyl-peroxy(hexane) or dibenzoyl peroxide.

The azo compounds used are essentially 2,2′-azobis(isobutyronitrile), 2,2′-azobis(2,4-dimethylvaleronitrile) and 2,2′-azobis(amidinopropyl)dihydrochloride (AIBA, corresponds to V-50™ from Wako Chemicals), 1,1′-azobis(1-cyclohexanecarbonitrile), 2,2′-azobis(2-amidinopropane)salts, 4,4′-azobis(4-cyanovaleric acid) or 2-(carbamoylazo)isobutyronitrile.

Suitable oxidizing agents for redox initiator systems are essentially the abovementioned peroxides. Corresponding reducing agents which may be used are sulfur compounds with a low oxidation state, such as alkali metal sulfites, for example potassium and/or sodium sulfite, alkali metal hydrogensulfites, for example potassium and/or sodium hydrogen sulfite, alkali metal metabisulfites, for example potassium and/or sodium metabisulfite, formaldehyde sulfoxylates, for example potassium and/or sodium formaldehyde sulfoxylate, alkali metal salts, specifically potassium and/or sodium salts, of aliphatic sulfinic acids and alkali metal hydrogen sulfides, such as, for example, potassium and/or sodium hydrogen sulfide, salts of polyvalent metals, such as iron(II) sulfate, iron(II) ammonium sulfate, iron(II) phosphate, enediols, such as dihydroxymaleic acid, benzoin and/or ascorbic acid, and reducing saccharides, such as sorbose, glucose, fructose and/or dihydroxyacetone.

The initiators are usually used in amounts up to 10% by weight, preferably 0.02 to 5% by weight, based on the monomers to be polymerized.

### Surfactants and Protective Colloids

The emulsion polymerization is usually carried out in the presence of surfactants and/or protective colloids.

During the preparation of the polymers used in the preparations according to the invention, at least one dispersion auxiliary is co-used which can keep both the monomer droplets and also polymer particles in dispersed distribution in the aqueous phase and thus ensures the stability of the aqueous polymer dispersion produced. Suitable as such are both the protective colloids customarily used for carrying out free-radical aqueous emulsion polymerizations and also emulsifiers.

Suitable protective colloids are, for example, polyvinyl alcohols, cellulose derivatives or polymers comprising vinylpyrrolidone. A detailed description of further suitable protective colloids is given in Houben-Weyl, Methoden der organischen Chemie [Methods of Organic Chemistry], Volume XIV/1, Makromolekulare Stoffe [Macromolecular Substances], pages 411 to 420, Georg-Thieme-Verlag, Stuttgart, 1961.

It is of course also possible to use mixtures of emulsifiers and/or protective colloids. The dispersion auxiliaries used are preferably exclusively emulsifiers whose relative molecular weights are usually below 1000, in contrast to the protective colloids. They can either be anionic, cationic or nonionic in nature. In cases where mixtures of interface-active substances are used, the individual components must of course be compatible with one another, which can be checked using a few preliminary experiments in cases of doubt. In general, anionic emulsifiers are compatible with one another and with nonionic emulsifiers. The same is also true for cationic emulsifiers, whereas anionic and cationic emulsifiers are in most cases not compatible with one another.

Customary emulsifiers are, for example, ethoxylated mono-, di- and trialkylphenols (degree of EO: 3 to 50, alkyl radical: C₄ to C₁₂), ethoxylated fatty alcohols (degree of EO: 3 to 50; alkyl radical: C₈ to C₃₆), and alkali metal and ammonium salts of alkyl sulfates (alkyl radical: C₈ to C₁₂), of sulfuric half-esters of ethoxylated alkanols (degree of EO: 4 to 30, alkyl radical: C₁₂ to C₁₈) and ethoxylated alkylphenols (degree of EO: 3 to 50, alkyl radical: C₄ to C₁₂), of alkylsulfonic acids (alkyl radical: C₁₂ to C₁₈) and of alkylarylsulfonic acids (alkyl radical: C₉ to C₁₈). Further suitable emulsifiers are given in Houben-Weyl, Methoden der organischen Chemie [Methods of Organic Chemistry], Volume XIV/1, Makromolekulare Stoffe [Macromolecular Substances], pages 192 to 208, Georg-Thieme-Verlag, Stuttgart, 1961.

Interface-active substances which have proven useful are also compounds of the general formula II

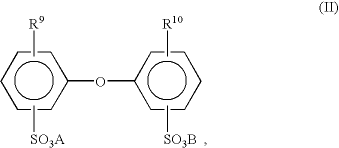

in which R⁹ and R¹⁰ are C₄-C₂₄-alkyl and one of the radicals R⁹ or R¹⁰ can also be hydrogen, and A and B may be alkali metal ions and/or ammonium ions. In the general formula II, R⁹ and R¹⁰ are preferably linear or branched alkyl radicals having 6 to 18 carbon atoms, in particular having 6, 12 and 16 carbon atoms or H atoms, where R⁹ and R¹⁰ are not both H atoms at the same time. A and B are preferably sodium, potassium or ammonium ions, particular preference being given to sodium ions. Compounds II in which A and B are sodium ions, R⁹ is a branched alkyl radical having 12 carbon atoms and R¹⁰ is an H atom or R⁹ are particularly advantageous. Use is often made of technical-grade mixtures which have a content of from 50 to 90% by weight of the monoalkylated product, for example Dowfax® 2A1 (trademark of Dow Chemical Company). The compounds II are generally known, e.g. from U.S. Pat. No. 4,269,749, and are commercially available.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates, alkyl glycol alkoxylates and diglycol alkoxylates and alkyl ether carboxylates can have between 1 and 10 ethylene oxide or propylene oxide units, preferably 1 to 3 ethylene oxide units, in the molecule.

Suitable nonionic surfactants are, for example, the reaction products of aliphatic alcohols or alkylphenols having 6 to 20 carbon atoms in the alkyl chain which may be linear or branched, with ethylene oxide and/or propylene oxide. The amount of alkylene oxide is about 6 to 60 moles per mol of alcohol. Also suitable are alkylamine oxides, mono- or dialkylalkanolamides, fatty acid esters of polyethylene glycols, ethoxylated fatty acid amides, alkyl polyglycosides, alkyl glycol alkoxylates and diglycol alkoxylates or sorbitan ether esters.

Suitable surfactants are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

As a rule, the amount of used dispersion auxiliaries is 0.1 to 5% by weight, preferably 1 to 3% by weight, in each case based on the total amount of the monomers to be polymerized by free radical means. It is often favorable if some or all of the dispersion auxiliary is added to the fluid reaction medium before the free-radical polymerization is initiated. Moreover, some or all of the dispersion auxiliary can be introduced into the reaction medium advantageously also together with the monomers to be polymerized, in particular in the form of an aqueous monomer emulsion during the polymerization. Preference is given to a combination of ionic and nonionic dispersion auxiliaries.

### Regulators

The regulators used are preferably alkanethiols. Mixtures of two or more regulators may also be used.

The alkanethiols used are linear and branched alkanethiols with a carbon chain length of C₁₀ to C₂₂. Particular preference is given to linear alkanethiols, and further preference to alkanethiols with a chain length of from C₁₂ to C₂₂, in particular from C₁₂ to C₁₈. Preferred alkanethiols are n-decanethiol, n-dodecanethiol, tert-dodecanethiol, n-tetradecanethiol, n-pentadecanethiol, n-hexadecanethiol, n-heptadecanethiol, n-octadecanethiol, n-nonadecanethiol, n-eicosanethiol, n-docosanethiol. Particular preference is given to linear, even-number alkanethiols The alkanethiols may also be used in mixtures.

The alkanethiols are usually used in amounts of from 0.1 to 5% by weight, in particular 0.25 to 2% by weight, based on the monomers to be polymerized. The alkanethiols are usually added to the polymerization together with the monomers.

Particular preference is given to using n-dodecanethiol as regulator.

### Hydrogen Peroxide Treatment

If, in the polymerization, alkanethiols with a carbon chain length of from C₁₀ to C₁₃ are used, a subsequent hydrogen peroxide treatment is in some cases required in order to obtain polymers with a neutral odor. For this hydrogen peroxide treatment which follows the polymerization, use is usually made of from 0.01 to 2.0% by weight, in particular 0.02 to 1.0% by weight, preferably 0.3 to 0.8% by weight, further preferably 0.03 to 0.15% by weight, of hydrogen peroxide, based on the monomers to be polymerized. It has proven advantageous to carry out the hydrogen peroxide treatment at a temperature of from 20 to 100° C., in particular from 30 to 80° C. The hydrogen peroxide treatment is usually carried out for a period from 30 min to 240 min, in particular from 45 min to 90 min.

If alkanethiols with a carbon chain length of from C₁₄ to C₂₂ are used, the hydrogen peroxide treatment can be omitted. In a further embodiment of the invention, however, a hydrogen peroxide treatment may follow even when using alkanethiols with a chain length of from C₁₄ to C₂₂.

### K Value

In a preferred embodiment, the K value of the polymers according to the invention is in the range from 20 to 70, particularly preferably in the range from 20 to 55 and very particularly preferably in the range from 25 to 45. The K value desired in each case can be adjusted through the choice of the polymerization conditions, for example the polymerization temperature and the initiator concentration.

In a preferred embodiment, regulators are used to adjust the K value, in particular when using emulsion and suspension polymerization.

The K value can be adjusted through the choice of the type and/or the amount of regulator. In a preferred embodiment, lower K values are established through relatively large amounts of regulator based on the total amount of monomer.

### Carrying out the Emulsion Polymerization

The emulsion polymerization usually takes place with the exclusion of oxygen, for example under a nitrogen or argon atmosphere, at temperatures in the range from 20 to 200° C. Polymerization temperatures in the range from 50 to 130° C., in particular 70 to 95° C., are advantageous.

In the case of free-radically initiated emulsion polymerization, in order to avoid coagulation, it is to be ensured, particularly at relatively high temperatures, that the polymerization mixture does not boil. This may be avoided, for example, by carrying out the polymerization reaction at an inert gas pressure which is higher than the vapor pressure of the polymerization mixture, for example 1.2 bar, 1.5 bar, 2 bar, 3 bar, 5 bar, 10 bar or even higher (in each case absolute values). The polymerization can be carried out batchwise, semicontinuously or continuously. The polymerization and the monomer and regulator feed are often carried out semicontinuously by the feed method.

The amounts of monomers and dispersant are expediently chosen such that a 30 to 80% strength by weight dispersion of the copolymers is obtained. Preferably, at least some of the monomers, initiators and, if appropriate, regulators are metered into the reaction vessel uniformly throughout the polymerization (feed procedure). However, it is also possible to have an initial charge of the monomers and the initiator in the reactor and to polymerize them, with cooling if appropriate.

According to a preferred embodiment, the polymerization is carried out using a seed latex. The seed latex is expediently prepared from the polymers to be polymerized in the first polymerization phase in the customary manner. The remainder of the monomer mixture is subsequently added, preferably by the feed method.

The polymerization reaction advantageously takes place until the monomer conversion is >95% by weight, preferably >98% by weight or >99% by weight.

It is often useful if the aqueous polymer dispersion obtained is subjected to an after-polymerization step in order to reduce further the amount of unreacted monomer. This measure is known to the person skilled in the art (for example EP-B 3957, EP-B 28348, EP-B 563726, EP-A 764699, EP-A 767180, DE-A 3718520, DE-A 3834734, DE-A 4232194, DE-A 19529599, DE-A 19741187, DE-A 19839199, DE-A 19840586, WO 95/33775 or U.S. Pat. No. 4,529,753).

### Processing the Dispersions

The aqueous polymer dispersions obtainable according to the invention can be converted to redispersible polymer powders in a simple manner.

If the polymer is prepared by emulsion polymerization, the dispersion obtained can either be incorporated directly into the aqueous, aqueous-alcoholic or alcoholic hair cosmetic preparation, or drying of the dispersion, e.g. spray-drying, fluidized spray drying, drum drying or freeze-drying, takes place, so that the polymer can be used and processed in the form of powder.

Preference is given to using spray drying. The polymer dried powders obtained in this way can advantageously be converted into an aqueous solution or dispersion again by dissolution or redispersion in water.

Pulverulent copolymers have the advantage of better storage properties, easier transportation option and generally exhibit a lower tendency to microbial attack. These include, quite generally, anionic, cationic, amphoteric and neutral polymers.

It is of course also possible to subject the aqueous polymer dispersion obtained to an inert-gas and/or steam stripping, likewise known to the person skilled in the art, before or after the after-polymerization step. This stripping operation preferably takes place after the after-polymerization step. As is described in EP-A 805169, partial neutralization of the dispersion to a pH in the range from 5 to 7, preferably to a pH in the range from 5.5 to 6.5, is advantageous before the physical deodorization.

Aqueous buffer solutions, such as, for example, buffers based on alkali metal and/or ammonium carbonate or bicarbonate are suitable for the neutralization.

The neutralizing agents are preferably added to the polymer dispersion as dilute aqueous solution.

The pH can, if appropriate, also be adjusted by adding a buffer solution, preference being given to buffers based on alkali metal or ammonium carbonate or hydrogencarbonate.

### Neutralizaton

Moreover, the polymers present in aqueous dispersion before or after the after-treatment can be partially or completely neutralized.

For using the polymers in hair cosmetic preparations in particular, partial or complete neutralization of the polymer dispersions is advantageous.

In preferred embodiments, the polymers are, for example, neutralized to at least 10, preferably to at least 30, further preferably to at least 40, particularly preferably to at least 50, very particularly preferably to at least 70 and especially to at least 95%.

In a particularly preferred embodiment, the polymers are neutralized to at least 99%. Neutralization to at least 100% is most preferred.

It is further advantageous if the neutralizing agent is added in more than the equivalent amount, equivalent amount being understood as meaning the amount which is needed to neutralize all of the neutralizable groups of the polymers.

The neutralization can also take place with
a mono-, di- or trialkanolamine having 2 to 5 carbon atoms in the alkanol radical, which is present in etherified form if appropriate, for example mono-, di- and triethanolamine, mono-, di- and tri-n-propanolamine, mono-, di- and triiso-propanolamine, 2-amino-2-methylpropanol and di(2-methoxyethyl)amine,
an alkanediolamine having 2 to 5 carbon atoms, for example 2-amino-2-methylpropane-1,3-diol and 2-amino-2-ethylpropane-1,3-diol, or
a primary, secondary or tertiary alkylamine having a total of 5 to 10 carbon atoms, for example N,N-diethylpropylamine or 3-diethylamino-1-propylamine.

Suitable alkali metal hydroxides for the neutralization are primarily sodium hydroxide, or potassium hydroxide and ammonium hydroxide.

Good neutralization results are often obtained with 2-amino-2-methylpropanol, triiso-propanolamine, 2-amino-2-ethylpropane-1,3-diol, N,N-dimethylaminoethanol or 3-diethylamino-1-propylamine.

Silicone polymers comprising amino groups are also particularly suitable for neutralizing the polymers in the preparations and compositions according to the invention. Suitable amino-containing silicone polymers are, for example, the silicone-aminopolyalkylene oxide block copolymers of WO 97/329171 the products Silsoft® A-843 (dimethicone bisamino hydroxypropyl copolyol) and Silsoft® A-858 (trimethylsilyl amodimethicone copolymer) (both Witco). Also suitable are, furthermore, the neutralization polymers of EP-A 1035144 and in particular the silicone-containing neutralization polymers of claim 12 of EP-A 1035144.

### Preserving the Dispersions

Customary preservatives are used for stabilizing and preserving the dispersion polymers. Preference is given to using hydrogen peroxide.

### Cosmetic and Pharmaceutical Preparations

The invention provides cosmetic and pharmaceutical preparations comprising the copolymers according to the invention.

The above-described copolymers present in the preparations according to the invention are exceptionally suitable for preparing cosmetic and pharmaceutical compositions. They serve here, for example, as polymeric film formers in preparations for body care, which includes use in cosmetic preparations for keratin surfaces such as skin, hair, nails, and also mouth care preparations.

They can be used and formulated universally in a very wide variety of cosmetic preparations and are compatible with customary components. In particular, the copolymers according to the invention are suitable for preparing hair cosmetic compositions.

Compared with customary polymers known from the prior art, they are advantageously suitable for producing elastic hair styles coupled with strong hold even at high atmospheric humidity.

The copolymers according to the invention are additionally characterized by good propellant gas compatibility, good solubility in water or aqueous/alcoholic solvent mixtures, suitability for use in low-VOC formulations and good ability to be washed out. In addition they also have good conditioning properties, i.e. they improve hair treated therewith in its sensorially ascertainable properties such as feel, volume, handlability etc.

Hair spray formulations based on the copolymers according to the invention are characterized by good rheological properties, such as, for example, flexural strength and elasticity and good sprayability. In addition, the films which can be produced from the polymers according to the invention are non-tacky.

The cosmetic preparations according to the invention can be in the form of aqueous or aqueous-alcoholic solutions, O/W and W/O emulsions in the form of shampoos, creams, foams, sprays (pump spray or aerosol), gels, gel sprays, lotions or mousse and accordingly are formulated with customary further auxiliaries.

The polymers are preferably formulated in hair cosmetic preparations as sprays (pump spray or aerosol). They are particularly preferably provided as VOC-55 formulations.

### Cosmetically or Pharmaceutically Acceptable Carrier B)

The preparations according to the invention additionally have at least one cosmetically or pharmaceutically acceptable carrier B which is chosen from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di or trihydric alcohols which are different from iii),
v) saturated acyclic and cyclic hydrocarbons,
vi) fatty acids,
vii) fatty alcohols,
viii) propellant gases,
   and mixtures thereof.

### Additives

The preparations according to the invention have, for example, an oil or fat component B) which is chosen from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons; animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl paimitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅ alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol, Preferred cyclic siloxanes have 4- to 8-membered rings. Suitable cyclic siloxanes are available commercially, for example under the name cyclomethicone.

Preferred oil and fat components B) are chosen from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, lard, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candelilla wax, spermaceti, and mixtures of the abovementioned oil and/or fatty components.

Suitable cosmetically and pharmaceutically compatible oil and/or fat components B) are described in Karl-Heinz Schrader, Grundlagen und Rezepturen der Kosmetika [Fundamentals and Formulations of Cosmetics], 2nd edition, Verlag Hüthig, Heidelberg, pp. 319-355, to which reference is made here.

Suitable hydrophilic carriers B) are chosen from water, mono-, di- or polyhydric alcohols having preferably 1 to 8 carbon atoms, such as ethanol, n-propanol, iso-propanol, propylene glycol, glycerol, sorbitol, etc.

The cosmetic compositions according to the invention may be skin cosmetic, hair cosmetic, pharmaceutical, hygienic or pharmaceutical compositions. On account of their film-forming properties, the above-described copolymers are particularly suitable as additives for hair and skin cosmetics.

Preferably, the compositions according to the invention are present in the form of sprays, gels, foam, ointment, cream, emulsion, suspension, lotion, milk or paste. If desired, liposomes or microspheres may also be used.

In a preferred embodiment of the invention, the compositions have a fraction of volatile organic components of at most 80% by weight, preferably at most 55% by weight and in particular at most 30% by weight. A preferred subject-matter is thus compositions which correspond to the low-VOC standard, i.e. VOC-80 and VOC-55 standard.

The cosmetically or pharmaceutically active compositions according to the invention can additionally comprise cosmetically and/or pharmaceutically active ingredients and auxiliaries.

Preferably, the cosmetic or pharmaceutical compositions according to the invention comprise at least one copolymer as defined above (=component A), at least one carrier B) as defined above and at least one constituent different therefrom which is chosen from cosmetic care and active ingredients, such as AHA acids, fruit acids, ceramides, phytantriol, collagen, vitamins and provitamins, for example vitamin A, E and C, retinol, bisabolol, panthenol, emulsifiers and coemulsifiers, surfactants, preservatives, perfume oils, thickeners, hair polymers, hair and skin conditioners, graft polymers, water-soluble or dispersible silicone-containing polymers, silicone compounds, natural and synthetic photoprotective agents, bleaches, gel formers, care agents, colorants, opacifiers, tinting agents, tanning agents, dyes, pigments, micropigments, such as titanium oxide or zinc oxide, consistency regulators, humectants, refatting agents, collagen, protein hydrolysates, lipids, antioxidants, antifoams, antistats, emollients, solubility promoters, repellents, superfatting agents, pearlescent waxes, consistency regulators, thickeners, solubilizers, complexing agents, pH regulators, reflectors, proteins and protein hydrolysates (e.g. wheat, almond or pea proteins) and softeners.

The auxiliaries may be present during the preparation of the polymers according to the invention and/or be added after the polymerization.

Examples of the particular classes of auxiliaries are given below without limiting the possible auxiliaries to those given by way of example.

Accordingly, the invention further provides the use of the acrylate polymers according to the invention in cosmetic, in particular hair cosmetic, preparations.

### Emulsifiers

Suitable emulsifiers are, for example, nonionogenic surfactants from at least one of the following groups:
(1) addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
(2) C12/18-fatty acid monoesters and diesters of addition products of from 1 to 30 mol of ethylene oxide onto glycerol;
(3) glycerol monoesters and diesters and sorbitan monoesters and diesters of saturated and unsaturated fatty acids having 6 to 22 carbon atoms and the ethylene oxide addition products thereof;
(4) alkyl monoglycosides and oligoglycosides having 8 to 22 carbon atoms in the alkyl radical and ethoxylated analogs thereof;
(5) addition products of from 15 to 60 mol of ethylene oxide onto castor oil and/or hydrogenated castor oil;
(6) polyol and, in particular polyglycerol, esters, such as, for example, polyglycerol polyricinoleate, polyglycerol poly-12-hydroxystearate or polyglycerol dimerate. Likewise suitable are mixtures of compounds from two or more of these classes of substance;
(7) addition products of from 2 to 15 mol of ethylene oxide onto castor oil and/or hydrogenated castor oil;
(8) partial esters based on linear, branched, unsaturated or saturated C_{6/22}-fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose);
(9) mono-, di- and trialkyl phosphates, and mono-, di- and/or Tri-PEG alkyl phosphates and salts thereof;
(10) wool wax alcohols;
(11) polysiloxane-polyalkyl-polyether copolymers and corresponding derivatives;
(12) mixed esters of pentaerythritol, fatty acids, citric acid and fatty alcohol as in German patent 1165574 and/or mixed esters of fatty acids having 6 to 22 carbon atoms, methylglycose and polyols, preferably glycerol or polyglycerol, and
(13) polyalkylene glycols.

The addition products of ethylene oxide and/or of propylene oxide onto fatty alcohols, fatty acids, alkyl phenols, glycerol monoesters and diesters, and sorbitan monoesters and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C₁₂-C₁₈-fatty acid monoesters and diesters of addition products of ethylene oxide onto glycerol are known from German patent 2024051 as refatting agents for cosmetic preparations. C₈-C₁₈-alkyl mono- and oligoglycosides, their preparation and their use are known from the prior art. They are prepared, in particular, by reacting glucose or oligosaccharides with primary alcohols having 8 to 18 carbon atoms. With regard to the glycoside ester, monoglycosides in which a cyclic sugar-radical is bonded to the fatty alcohol glycosidically, and also oligomeric glycosides having a degree of oligomerization up to preferably about 8 are suitable. The degree of oligomerization here is a statistical average value which is based on a homolog distribution customary for such technical-grade products.

It is also possible for the emulsifiers used to be zwitterionic surfactants. Zwitterionic surfactants is the term used to refer to those surface-active compounds which carry at least one quaternary ammonium group and at least one carboxylate and/or one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines having in each case 8 to 18 carbon atoms in the alkyl or acyl group, and cocoacylaminoethylhydroxyethylcarboxymethyl-glycinate.

Particular preference is given to the fatty acid amide derivative known under the CTFA name Cocamidopropyl Betaine. Likewise suitable emulsifiers are ampholytic surfactants. Ampholytic surfactants are understood as meaning those surface-active compounds which, apart from a C₈-C₁₈-alkyl or -acyl group in the molecule, comprise at least one free amino group and at least one —COOH and/or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C₁₂ to C₁₈-acylsarcosine.

Besides the ampholytic emulsifiers, quaternary emulsifiers are also suitable, particular preference being given to those of the ester quat type, preferably methyl-quaternized difatty acid triethanolamine ester salts.

### Oil Bodies

Suitable oil bodies are, for example, Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of hydroxycarboxylic acids with linear or branched C₆-C₂₂-fatty alcohols, in particular dioctyl 2-hydroxysuccinate, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, vegetable oils, branched primary alcohols, substituted cyclohexanes, linear and branched C₆-C₂₂-fatty alcohol carbonates, Guerbet carbonates, esters of benzoic acid with linear and/or branched C₆₋₂₂-alcohols (e.g. Finsolvâ TN), linear or branched, symmetrical or asymmetrical dialkyl ethers having 6 to 22 carbon atoms per alkyl group, ring-opening products of epoxidized fatty acid esters with polyols, silicone oils and/or aliphatic or naphthenic hydrocarbons.

### Preservatives

Examples of suitable preservatives are phenoxyethanol, formaldehyde solution, parabens, pentanediol or sorbic acid, and the other classes of substance listed in Appendix 6, Part A and B, of the Cosmetics Directive.

### Perfume Oils

If appropriate, the cosmetic preparations may comprise perfume oils. Perfume oils which may be mentioned are, for example, mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumene, juniper), fruit peels (bergamot, lemon, orange), roots (mace, angelica, celery, cardamon, costus, iris, calmus), woods (pinewood, sandalwood, guaiac wood, cedar wood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, 4-tert-butylcyclohexyl acetate, linalyl acetate, dimethylbenzylcarbinyl acetate, phenylethyl acetate, linalyl benzoate, benzyl formate, ethyl methylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, and the ketones include, for example, the ionones, cc-isomethylionene and methyl cedryl ketone, and the alcohols include anethol, citronellol, eugenol, isoeugenol, geraniol, linalool, phenylethyl alcohol and terpineol, and the hydrocarbons include mainly the terpenes and balsams. However, preference is given to using mixtures of different fragrances which together produce a pleasing scent note. Essential oils of lower volatility, which are mostly used as flavor components, are also suitable as perfume oils, e.g. sage oil, chamomile oil, oil of cloves, balm oil, mint oil, cinnamon leaf oil, lime blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamenaldehyde, linalool, boisambrene forte, ambroxan, indole, hedione, sandelice, lemon oil, mandarin oil, orange oil, allyl amyl glycolate, cyclovertal, lavandin oil, clarysage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, Romillat, Irotyl and Floramat alone or in mixtures.

### Superfatting Agents

Superfatting agents which may be used are substances such as, for example, lanolin and lecithin, and also polyethoxylated or acylated lanolin and lecithin derivatives, polyol fatty acid esters, monoglycerides and fatty acid alkanolamides, the latter also serving as foam stabilizers.

### Pearlescent Waxes

Examples of suitable pearlescent waxes are: alkylene glycol esters, specifically ethylene glycol distearate; fatty acid alkanolamides, specifically coconut fatty acid diethanolamide, partial glycerides, specifically stearic acid monoglyceride; esters of polybasic, optionally hydroxyl-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

### Bodying Agents

Suitable bodying agents are primarily fatty alcohols or hydroxy fatty alcohols having 12 to 22 and, preferably, 16 to 18, carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, polysaccharides, in particular xanthan gum, guar-guar, agar-agar, alginates and tyloses, carboxymethylcellulose and hydroxyethylcellulose, and also relatively high molecular weight polyethylene glycol monoesters and diesters of fatty acids, polyacrylates (e.g. Carbopol™ from Goodrich or Synthalen™ from Sigma), polyacryl-amides, polyvinyl alcohol and polyvinylpyrrolidone, surfactants, such as, for example, ethoxylated fatty acid glycerides, esters of fatty acids with polyols, such as, for example pentaerythritol or trimethylolpropane, fatty alcohol ethoxylates with a narrowed homolog distribution or alkyl oligoglucosides, and electrolytes, such as sodium chloride and ammonium chloride.

### Thickeners

Customary thickeners in such formulations are crosslinked polyacrylic acids and derivatives thereof, polysaccharides such as xanthan gum, agar-agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Suitable thickeners are also the Aculyn® grades from Rohm and Haas, such as Aculyn® 22 (copolymer of acrylates and methacrylic acid ethoxylates with stearyl radical (20 EO units)) and Aculyn® 28 (copolymer of acrylates and methacrylic acid ethoxylates with behenyl radical (25 EO units)).

### Hydrotropic Agents

To improve the flow behavior, it is also possible to use hydrotropic agents, such as, for example, ethanol, isopropyl alcohol or polyols. Polyols which are suitable here preferably have 2 to 15 carbon atoms and at least two hydroxyl groups.

Typical examples are
glycerol;
alkylene glycols, such as, for example, ethylene glycol, diethylene glycol, propylene glycol, butylene glycol, hexylene glycol, and polyethylene glycols with an average molecular weight of from 100 to 1000 daltons; technical-grade oligoglycerol mixtures with a degree of self-condensation of from 1.5 to 10, such as, for example, technical-grade diglycerol mixtures with a diglycerol content of from 40 to 50% by weight;
methylol compounds, such as, in particular, trimethylolethane, trimethylolpropane, trimethylolbutane, pentaerythritol and dipentaerythritol;
lower alkyl glucosides, in particular those having 1 to 8 carbon atoms in the alkyl radical, such as, for example, methyl- and butylglucoside;
sugar alcohols having 5 to 12 carbon atoms, such as, for example, sorbitol or mannitol;
sugars having 5 to 12 carbon atoms, such as, for example, glucose or sucrose;
amino sugars, such as, for example, glucamine.

UV Photoprotection

The photoprotective filters used in cosmetic preparations have the task of preventing harmful effects of sunlight on the human skin, or at least of reducing their consequences. In addition, however, these photoprotective filters also serve to protect further ingredients against decomposition or degradation by UV radiation. In hair cosmetic formulations the aim is to prevent damage to keratin fibers as a result of UV rays.

The sunlight which reaches the surface of the earth has a fraction of UV-B radiation (280 to 320 nm) and of UV-A radiation (320 to 400 nm) which directly border the visible light region. The effect on the human skin is evident particularly in the case of UV-B radiation through sunburn.

The maximum of the erythema activity of sunlight is given as the relatively narrow range around 308 nm.

To protect against UV-B radiation, numerous compounds are known, which are, inter alia, derivatives of 3-benzylidenecamphor, of 4-aminobenzoic acid, of cinnamic acid, of salicylic acid, of benzophenone, and of 2-phenylbenzimidazole.

It is also important to have available filter substances for the range between about 320 nm and about 400 nm, the so-called UV-A region, since its rays can cause reactions in photosensitive skin. It has been found that UV-A radiation leads to damage of the elastic and collagenous fibers of connective tissue, which causes the skin to age prematurely, and that it should be regarded as the cause of numerous phototoxic and photoallergic reactions. The harmful effect of UV-B radiation can also be intensified by UV-A radiation.

UV photoprotective filters which may be used are oil-soluble organic UV-A filters and/or UV-B filters and/or water-soluble organic UV-A filters and/or UV-B filters. The total amount of UV photoprotective filters is generally 0.1% by weight to 30% by weight, preferably 0.5 to 15% by weight, in particular 1 to 10% by weight, based on the total weight of the preparation.

The UV photoprotective filters are advantageously chosen such that the preparations protect the skin from the entire range of ultraviolet radiation.

Examples of UV photoprotective filters are:

| No. | Substance | CAS No. |
|---|---|---|
| 1 | 4-Aminobenzoic acid | 150-13-0 |
| 2 | 3-(4′-Trimethylammonium)benzylidenebornan-2-one | 52793-97-2 |
| | methylsulfate | |
| 3 | 3,3,5-Trimethylcyclohexyl salicylate (homosalate) | 118-56-9 |
| 4 | 2-Hydroxy-4-methoxybenzophenone (oxybenzone) | 131-57-7 |
| 5 | 2-Phenylbenzimidazole-5-sulfonic acid and its potassium, | 27503-81-7 |
| | sodium and triethanolamine salts | |
| 6 | 3,3′-(1,4-Phenylenedimethine)bis(7,7-dimethyl-2-oxobi- | 90457-82-2 |
| | cyclo[2.2.1]heptane-1-methanesulfonic acid) and its salts | |
| 7 | Polyethoxy ethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfo)benzylidenebornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 2,4,6-Trianilino(o-carbo-2′-ethylhexyl-1′-oxy)-1,3,5- | 88122-99-0 |
| | triazine | |
| 18 | 3-Imidazol-4-ylacrylic acid and its ethyl ester | 104-98-3 |
| 19 | Menthyl o-aminobenzoate or: 5-methyl-2-(1-methylethyl)- | 134-09-8 |
| | 2-aminobenzoate | |
| 20 | Glyceryl p-aminobenzoate or 1-glyceryl 4-aminobenzoate | 136-44-7 |
| 21 | 2,2′-Dihydroxy-4-methoxybenzophenone (dioxybenzone) | 131-53-3 |
| 22 | 2-Hydroxy-4-methoxy-4-methylbenzophenone | 1641-17-4 |
| | (mexenone) | |
| 23 | Triethanolamine salicylate | 2174-16-5 |
| 24 | Dimethoxyphenylglyoxalic acid or: sodium 3,4-dimethoxy- | 4732-70-1 |
| | phenylglyoxalate | |
| 25 | 3-(4′Sulfo)benzylidenebornan-2-one and its salts | 56039-58-8 |
| 26 | 2,2′,4,4′-Tetrahydroxybenzophenone | 131-55-5 |
| 27 | 2,2′-Methylenebis[6(2H-benzotriazol-2-yl)-4-(1,1,3,3,- | 103597-45-1 |
| | tetramethylbutyl)phenol] | |
| 28 | 2,2′-(1,4-Phenylene)bis-1H-benzimidazole-4,6-disulfonic | 180898-37-7 |
| | acid, Na salt | |
| 29 | 2,4-bis-[4-(2-Ethylhexyloxy)-2-hydroxy]phenyl-6-(4- | 187393-00-6 |
| | methoxyphenyl)-(1,3,5)-triazine | |
| 30 | 3-(4-Methylbenzylidene)camphor | 36861-47-9 |
| 31 | Polyethoxyethyl 4-bis(polyethoxy)paraaminobenzoate | 113010-52-9 |
| 32 | 2,4-Dihydroxybenzophenone | 131-56-6 |
| 33 | 2,2′-Dihydroxy-4,4′-dimethoxybenzophenone-5,5′- | 3121-60-6 |
| | disodium sulfonate | |

Further photoprotective agents which can be combined are, inter alia, the following compounds:

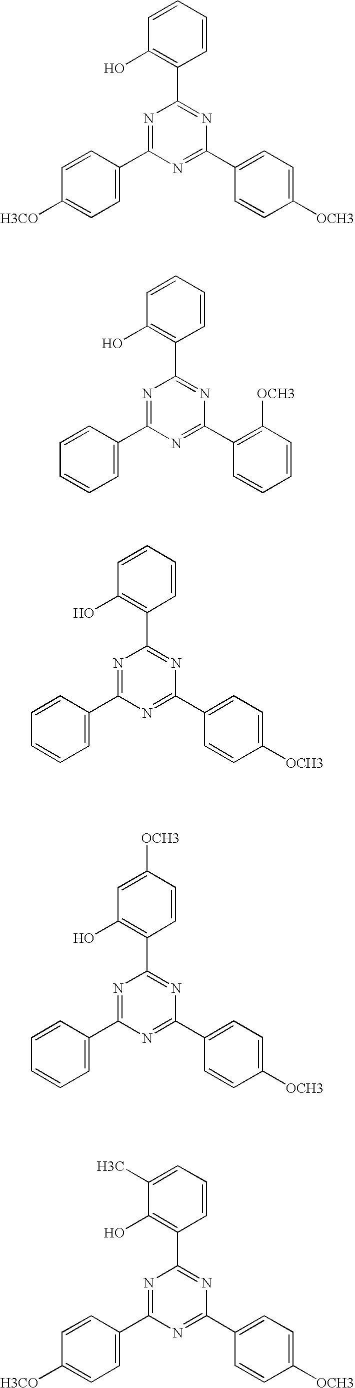

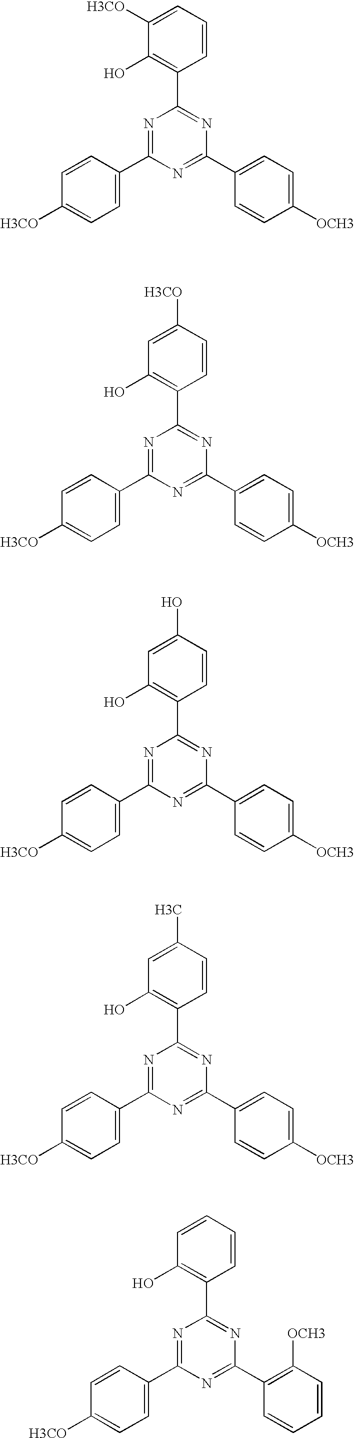

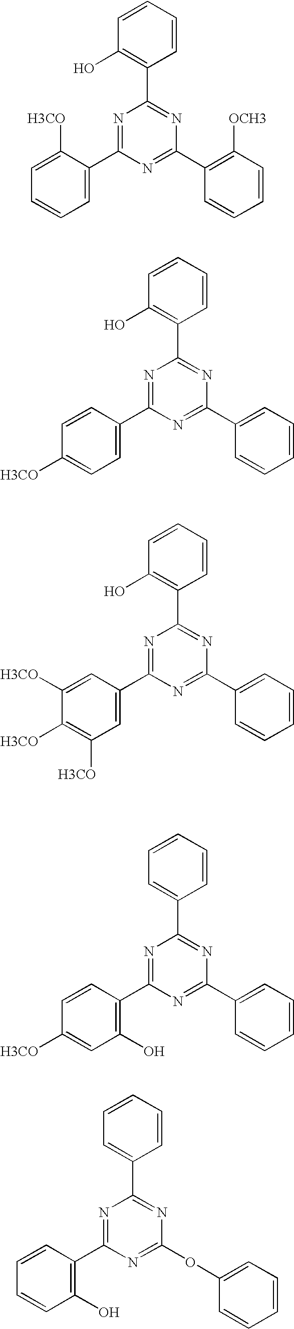

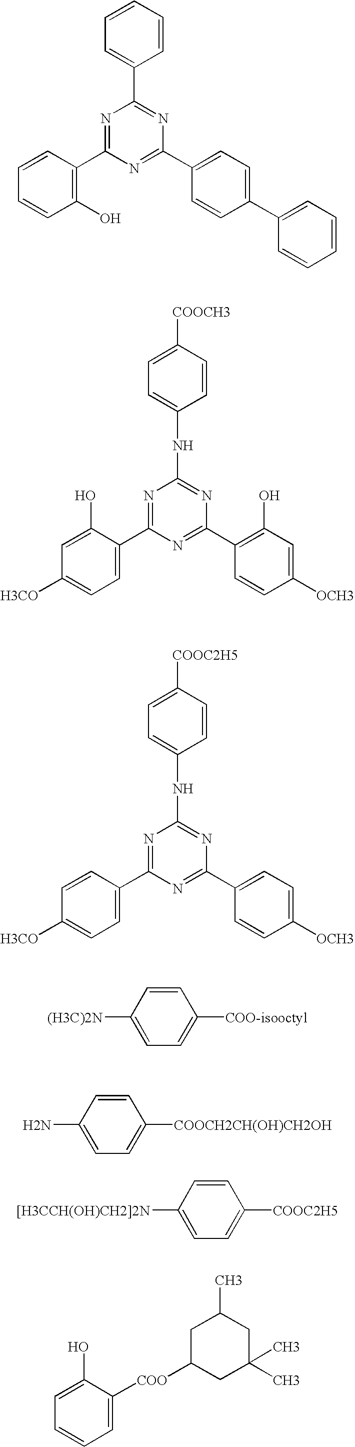

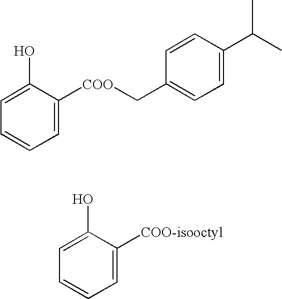

Also suitable are pigments which repel UV rays, such as titanium dioxide, talc and zinc oxide.

Photoprotective agents suitable for use in the preparations according to the invention are also the compounds specified in EP-A 1 084 696 in paragraphs [0036] to [0053], which is hereby incorporated in its entirety by reference.

The list of specified UV photoprotective filters which can be used in the preparations according to the invention is not of course intended to be limiting.

### Antibacterial Agents

In addition, antibacterial agents can also be used in the preparations according to the invention. These generally include all suitable preservatives with a specific action against gram-positive bacteria, e.g. triclosan (2,4,4′-trichloro-2′-hydroxydiphenyl ether), chlorhexidine (1,1′-hexamethylenebis[5-(4-chlorophenyl)biguanide) and TTC (3,4,4′ trichlorocarbanilide).

Quaternary ammonium compounds are in principle likewise suitable, but are used preferably for disinfecting soaps and washing lotions.

Numerous fragrances also have antimicrobial properties. Specific combinations having particular effectiveness against gram-positive bacteria are used for the composition of so-called deodorant perfumes.

Also, a large number of essential oils or characteristic ingredients thereof, such as, for example, oil of cloves (eugenol), mint oil (menthol) or thyme oil (thymol), exhibit marked antimicrobial effectiveness.

The antibacterially effective substances are generally used in concentrations of from about 0.1 to 0.3% by weight.

On account of their film-forming properties, the copolymers described above are particularly suitable as additives for hair and skin cosmetics.

Preferably, the compositions according to the invention are present in the form of a spray, gel, foam, ointment, cream, emulsion, suspension, lotion, milk or paste. If desired, liposomes or microspheres may also be used.

The cosmetically or pharmaceutically active compositions according to the invention can additionally comprise cosmetically and/or pharmaceutically active ingredients and auxiliaries.

Cosmetically and/or Pharmaceutically Active Ingredients

Further suitable cosmetically and/or pharmaceutically active ingredients are, for example, coloring active ingredients, skin and hair pigmentation agents, tinting agents, tanning agents, bleaches, keratin-hardening substances, repellent active ingredients, substances with a hyperemic effect, substances with keratolytic and keratoplastic activity, antidandruff active ingredients, antiphlogistics, substances with a keratinizing effect, active ingredients with an antioxidative or free-radical scavenging effect, skin-moisturizing or humectant substances, refatting active ingredients, antierythimatous or antiallergic active ingredients and mixtures thereof.

Active ingredients which tan the skin artificially and which are suitable for tanning the skin without natural or artificial irradiation with UV rays are, for example, dihydroxyacetone, alloxan and walnut shell extract.

Suitable keratin-hardening substances are generally active ingredients as are also used in antiperspirants, such as, for example, potassium aluminum sulfate, aluminum hydroxychloride, aluminum lactate, etc.

Antimicrobial active ingredients are used to destroy microorganisms or to inhibit their growth and thus serve both as preservatives and also as deodorizing substance which reduces the formation or the intensity of body odor. These include, for example, customary preservatives known to the person skilled in the art, such as p-hydroxybenzoate, imidazolidinylurea, formaldehyde, sorbic acid, benzoic acid, salicylic acid, etc. Such deodorizing substances are, for example, zinc ricinoleate, triclosan, undecylenic alkylolamides, triethyl citrate, chlorhexidine etc.

Suitable repellent active ingredients are compounds which are able to drive away or keep certain animals, in particular insects, away from humans. These include, for example, 2-ethyl-1,3-hexanediol, N,N-diethyl-m-toluamide etc.

Suitable substances with hyperemic activity, which stimulate the blood flow through the skin, are, for example, essential oils, such as dwarf pine, lavender, rosemary, juniper-berry, roast chestnut extract, birch leaf extract, hayseed extract, ethyl acetate, camphor, menthol, peppermint oil, rosemary extract, eucalyptus oil, etc.

Suitable substances with keratolytic and keratoplastic activity are, for example, salicylic acid, calcium thioglycolate, thioglycolic acid and its salts, sulfur, etc.

Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counter skin irritations, are, for example, allantoin, bisabolol, dragosantol, camomile extract, panthenol, etc.

The cosmetic compositions according to the invention can comprise at least one cosmetically or pharmaceutically acceptable polymer as cosmetic and/or pharmaceutical active ingredient (and also if appropriate as auxiliary).

### Hair Cosmetic Preparations

Possible forms of hair cosmetic preparations which may be mentioned are hair treatments, hair lotions, hair rinses, hair emulsions, split-end fluids, neutralizers for permanent waves, hot-oil treatment preparations, conditioners, curl relaxers, styling wrap lotions, setting lotions, shampoos, hair waxes, pomades, hair foams, hair colorants or hair sprays.

Particular preference is given to hair cosmetic preparations for setting hair styles which are in the form of spray preparations and/or hair foams.

The polymers present in the hair cosmetic preparations according to the invention are characterized by their high compatibility with the nonpolar propellants in spray preparations, in particular with hydrocarbons, such as n-propane, isopropane, n-butane, isobutane, n-pentane and mixtures thereof and in particular by the excellent sprayability as pump spray or aerosol.

The polymers are also very readily compatible with other additives customary in hair cosmetics, have a good hair-setting action, form films with very good mechanical properties, can be washed out easily and are characterized in that they virtually do not cause the hair to stick together.

Besides the freedom from odor, the polymers have excellent results for the performance properties in hair cosmetic preparations. They dissolve in alcohols such as ethanol or isopropanol and in mixtures of these alcohols with water to give clear solutions. The clarity of the solutions is retained even if the solutions are used in standard spray formulations together with propellants such as dimethyl ether. In particular, they give clear formulations in aqueous low-VOC preparations with at most 55% by weight of volatile organic constituents (VOC-55).

The hair cosmetic preparations according to the invention can be washed out of the hair completely. Hair treated therewith has increased suppleness and a pleasant natural feel. The setting action is simultaneously high here, meaning that in principle it is possible to reduce the required amount of film formers in the hair spray formulation On account of the freedom from odor of the polymers, it is possible to dispense with an addition of odor-concealing perfume oils.

The hair cosmetic preparations usually comprise 0.1 to 20% by weight, preferably 0.5 to 10% by weight, in particular 2 to 10% by weight, of the partially or completely neutralized polymers, based on the preparation.

### Hair Spray Formulations

A preferred embodiment of the invention are hair spray preparations which comprise the following constituents:
0.1 to 20% by weight, preferably 0.5 to 15% by weight, in particular 1 to 10% by weight of the partially or completely neutralized polymer
0 to 99.9% by weight, preferably 1 to 50% by weight, in particular 10 to 20% by weight, of water
0 to 95% by weight, preferably 20 to 60% by weight, in particular 25 to 50% by weight of a customary organic solvent such as primarily ethanol, isopropanol and dimethoxymethane and in addition also acetone, n-propanol, n-butanol, 2-methoxypropan-1-ol, n-pentane, n-hexane, cyclohexane, n-heptane, n-octane or dichloromethane or mixtures thereof
0 to 90% by weight, preferably 30 to 80% by weight, in particular 45 to 60% by weight, of a customary propellant, such as n-propane, isopropane, n-butane, isobutane, 2,2-dimethylbutane, n-pentane, isopentane, dimethyl ether, difluoroethane, fluorotrichloromethane, dichlorodifluoromethane or dichlorotetrafluoroethane, HFC 152 A or mixtures thereof.

Alkanolamines are used for neutralizing various types of acids and for establishing the pH of cosmetic products. Examples (INCI names) are Aminomethyl Propanol, Diethanolamine, Diisopropanolamine, Ethanolamine, Methylethanolamine, N-Lauryl Diethanolamine, Triethanolamine, Triisopropanolamine, etc. In addition, alkali metal hydroxides (e.g. NaOH, KOH) and other bases can be used for the neutralization (e.g. histidine, arginine, lysine or ethylenediamines, diethylenetriamine, melamine, benzoguanamine). All of the bases given can be used on their own or as a mixture with other bases to neutralize cosmetic products containing acid.

### Propellants (Propellant Gases)

Of the compounds specified, the propellants (propellant gases) used are primarily the hydrocarbons, in particular propane, n-butane, n-pentane and mixtures thereof, and also dimethyl ether and difluoroethane. If appropriate, one or more of said chlorinated hydrocarbons are co-used in propellant mixtures, but only in small amounts, for example up to 20% by weight, based on the propellant mixture.

The hair cosmetic preparations according to the invention are also particularly suitable for pump spray preparations without the addition of propellants, or else for aerosol sprays with customary compressed gases, such as nitrogen, compressed air or carbon dioxide as propellant.

The copolymers according to the invention are particularly suitable as setting agents in hair styling preparations, in particular hair sprays (aerosol sprays and pump sprays without propellant gas) and hair foams (aerosol foams and pump foams without propellant gas).

In a preferred embodiment, spray preparations comprise
a) 0.1 to 10% by weight of at least one copolymer according to the invention,
b) 20 to 99.9% by weight of water and/or alcohol,
c) 0 to 70% by weight of at least one propellant,
d) 0 to 20% by weight of further constituents.

Propellants are the propellants customarily used for hair sprays or aerosol foams. Preference is given to mixtures of propane/butane, pentane, dimethyl ether, 1,1-difluoroethane (HFC-152 a), carbon dioxide, nitrogen or compressed air,

A formulation for aerosol hair foams preferred according to the invention comprises
a) 0.1 to 10% by weight of at least one copolymer according to the invention,
b) 55 to 99.8% by weight of water and/or alcohol,
c) 5 to 20% by weight of a propellant,
d) 0.1 to 5% by weight of an emulsifier,
e) 0 to 10% by weight of further constituents.

Emulsifiers which can be used are all emulsifiers customarily used in hair foams. Suitable emulsifiers may be nonionic, cationic and/or anionic or amphoteric.

Propellants of particular suitability for aerosol foams are mixtures of dimethyl ether and, if appropriate halogenated, hydrocarbons, such as propane, butane, pentane or HFC-152 a.

Examples of nonionic emulsifiers (INCI nomenclature) are laureths, e.g. laureth-4; ceteths, e.g. cetheth-1, polyethylene glycol cetyl ether; ceteareths, e.g. cetheareth-25, polyglycol fatty acid glycerides, hydroxylated lecithin, lactyl esters of fatty acids, alkyl polyglycosides.

A water-containing standard spray formulation has, for example, the following composition:
2 to 10% by weight of the polymer neutralized to 100% with 2-amino-2-methylpropanol
10 to 76% by weight of ethanol
2 to 20% by weight of water
10 to 60% by weight of dimethyl ether and/or propane/n-butane and/or propane/isobutane.

In a preferred embodiment, the hair cosmetic formulations according to the invention comprise
a) 0.05 to 20% by weight of at least one copolymer according to the invention,
b) 20 to 99.95% by weight of water and/or alcohol,
c) 0 to 50% by weight of at least one propellant gas,
d) 0 to 5% by weight of at least one emulsifier,
e) 0 to 3% by weight of at least one thickener, and
f) up to 25% by weight of further constituents.

Alcohol is understood as meaning all alcohols customary in cosmetics, e.g. ethanol, isopropanol, n-propanol.

### Further Polymers

For the targeted setting of the properties of the hair cosmetic preparations, it may be advantageous to use the setting polymers in a mixture with other polymers. Suitable conventional polymers for this purpose are, for example, anionic, cationic, amphoteric and neutral polymers.

Preferred examples of such other polymers are
copolymers of ethyl acrylate and methacrylic acid
copolymers of N-tert-butylacrylamide, ethyl acrylate and acrylic acid
polyvinylpyrrolidones
polyvinylcaprolactams
polyurethanes
copolymers of acrylic acid, methyl methacrylate, octylacrylamide, butylaminoethyl methacrylate and hydroxypropyl methacrylate,
copolymers of vinyl acetate and crotonic acid and/or (vinyl)neodecanoate,
copolymers of vinyl acetate and/or vinyl propionate and N-vinylpyrrolidone,
carboxyfunctional copolymers of vinylpyrrolidone, t-butyl acrylate, methacrylic acid,
copolymers of tert-butyl acrylate, methacrylic acid and dimethicone copolyol.

Surprisingly, it has been found that preparations which comprise the polymers in combination with these other polymers have unexpected properties. The preparations according to the invention are superior to the preparations of the prior art particularly with regard to their hair cosmetic properties. In addition, they have very good film-forming and setting properties.

Copolymers of ethyl acrylate and methacrylic acid (INCI name: Acrylates Copolymer), are obtainable, for example, as commercial products Luviflex® Soft (BASF).

Copolymers of N-tert-butylacrylamide, ethyl acrylate and acrylic acid (INCI name: Acrylates/Acrylamide Copolymer) are available, for example, as commercial products Ultrahold®Strong, Ultrahold®8 (BASF).

Polyvinylpyrrolidones (INCI name: PVP) are available, for example, under the trade names Luviskol®K, Luviskol®K 30™ (BASF) and PVP K (ISP).

Polyvinylcaprolactams (INCI: Polyvinylcaprolactams) are obtainable, for example, under the trade name Luviskol®Plus™ (BASF).

Polyurethanes (INCI: Polyurethane-1) are available, for example, under the trade name Luviset®PUR.

Copolymers of acrylic acid, methyl methacrylate, octylacrylamide, butylaminoethyl methacrylate, hydroxypropyl methacrylate (INCI: Octylacrylamide/Acrylates/Butylaminoethyl Methacrylate Copolymer) are known, for example, under the trade names Amphomer®28-4910 and Amphomer®LV-71 (National Starch).

Copolymers of vinyl acetate and crotonic acid (INCI: VA/Crotonate/Copolymer) are available, for example, under the trade names Luviset CA 66® (BASF), Resyn®28-1310 (National Starch) and Aristoflex®A (Celanese).

Copolymers of vinyl acetate, crotonic acid and (vinyl)neodecanoate (INCI: VA/Crotonates/Neodecanoate Copolymer) are available, for example, under the trade names Resyn®28-2930 (National Starch) und Luviset®CAN (BASE).

Copolymers of vinyl acetate and N-vinylpyrrolidone (INCI: PVP/VA) are available, for example, under the trade names Luviskol VA® (BASF) and PVP/VA (ISP).

Carboxy functional copolymers of vinylpyrrolidone, t-butyl acrylate, methacrylic acid are available, for example, under the trade name Luviskol®VBM (BASF).

Copolymers of tert-butyl acrylate, methacrylic acid and dimethicone copolyol are available, for example, under the trade name Luviflex®Silk (BASF).

Suitable further polymers are, for example, anionic polymers. Such anionic polymers are homopolymers and copolymers of acrylic acid and methacrylic acid different from the (meth)acrylate polymers according to the invention, or salts thereof, copolymers of acrylic acid and acrylamide and salts thereof, sodium salts of polyhydroxycarboxylic acids, copolymers of acrylic acid and methacrylic acid with, for example, hydrophobic monomers, e.g. C₄-C₃₀-alkyl esters of (meth)acrylic acid, C₄-C₃₀-alkylvinyl esters, C₄-C₃₀-alkyl vinyl ethers and hyaluronic acid, and further polymers known under the trade names Amerhold DR-25, Ultrahold®, Luviset®P.U.R., Acronal®, Acudyne®, Lovocryl®, Versatyl®, Amphomer® (28-4910, LV-71), Placise® L53, Gantrez®ES 425, Advantage Plus®, Omnirez®2000, Resyn®28-1310, Resyn®28-2930, Balance® (0/55), Acudyne®255, Aristoflex®A or Eastman AQ®.

Suitable additional polymers may also be water-soluble or water-dispersible polyesters, polyureas, copolyurethane ureas, maleic anhydride copolymers optionally reacted with alcohols, or anionic polysiloxanes.

Additional further suitable polymers are, for example, also cationic polymers with the INCI name Polyquaternium, such as, for example,
copolymers of N-vinylpyrrolidone/N-vinylimidazolium salts (available, for example, under the trade names Luviquat®FC, Luviquat®HM, Luviquat® MS, Luviquat®Care (BASF),
copolymers of N-vinylcaprolactam/N-vinylpyrrolidone/N-vinylimidazolium salts (available, for example, under the trade name Luviquat®Hold), copolymers of N-vinylpyrrolidone/dimethylaminoethyl methacrylate, quaternized with diethyl sulfate (available, for example, under the trade name Luviquat®PQ11),
cationic cellulose derivatives (Polyquaternium-4 and -10),
acrylamide copolymers (Polyquaternium-7),
Styleeze®CC-10, Aquafex®SF-40,
guar hydroxypropyltrimethylammonium chloride (INCI: Hydroxypropyl Guar Hydroxypropyltrimonium Chloride),
polyethyleneimines and salts thereof,
polyvinylamines and salts thereof.

Suitable further hair cosmetic polymers are also neutral polymers such as polyvinylpyrrolidone, copolymers of N-vinylpyrrolidone and vinyl acetate and/or vinyl propionate, polysiloxanes, polyvinylcaprolactam and copolymers with N-vinylpyrrolidone, cellulose derivatives, polyaspartic acid salts and derivatives. These include those known under the trade names Luviskol® (K, VA, Plus), PVP K, PVP/VA, Advantage®HC and H₂OLD® EP-1.

Also suitable are, furthermore, biopolymers, i.e. polymers which are obtained from naturally renewable raw materials and are constructed from natural monomer building blocks, e.g. cellulose derivatives, chitin, chitosan, DNA, hyaluronic acid and RNA derivatives.

Further suitable polymers are also betainic polymers, such as Yukaformers (R205, SM) and Diaformers.

Of course, if required, all of the abovementioned cosmetic ingredients can also be added to the hair cosmetic preparations.

In a further preferred embodiment of the invention, the cosmetic preparation is a styling product.

A preparation suitable according to the invention for styling gels can, for example, having the following composition:
a) 0.1 to 10% by weight of at least one copolymer according to the invention,
b) 80 to 99.85% by weight of water and/or alcohol,
c) 0 to 3% by weight, preferably 0.05 to 2% by weight, of a gel former,
d) 0 to 20% by weight of further constituents.

The use of gel formers may, however, be advantageous in order to establish specific rheological or other performance properties of the gels. Gel formers which can be used are all gel formers customary in cosmetics. These include slightly crosslinked polyacrylic acid, for example carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCI), sodium acrylate copolymers (and) Paraffinum Liquidum (and) PPG-1 trideceth-6, acrylamidopropyltrimonium chloride/acrylamide copolymers, steareth-10 alkyl ether acrylate copolymers, polyquaternium-37 (and) Paraffinum Liquidum (and) PPG-1 trideceth-6, polyquaternium 37 (and) propylene glycol dicapratedicaprylate (and) PPG-1 trideceth-6, polyquaternium-7, polyquaternium-44.

A further preferred embodiment of the invention are products for the hair which are chosen from the hair-washing compositions and products for the hair which are rinsed out or not rinsed out and are applied before or after hair washing, coloring, bleaching, permanent waving or straightening.

In a further preferred embodiment of the invention, the cosmetic preparation is formulated in an atomizer, pump bottle or aerosol container in order to obtain a spray, a lacquer or a foam.

The invention further provides a method of treating hair wherein the hair is brought into contact with the cosmetic preparation according to the invention and, if appropriate, is rinsed with water.

The copolymers according to the invention as defined above can preferably be used in shampoo formulations as setting and/or conditioning agents. Preferred shampoo formulations comprise
a) 0.05 to 10% by weight of at least one copolymer according to the invention,
b) 25 to 94.95% by weight of water,
c) 5 to 50% by weight of surfactants,
c) 0 to 5% by weight of a further conditioning agent,
d) 0 to 10% by weight of further cosmetic constituents.

In the shampoo formulations it is possible to use all anionic, neutral, amphoteric or cationic surfactants used customarily in shampoos.

The invention further provides the use of the copolymer according to the invention in cosmetic preparations.

The invention further provides the use of the copolymer according to the invention as auxiliary in pharmacy, preferably as or in (a) coating composition(s) for solid drug forms, for modifying rheological properties, as surface-active compound, as or in (an) adhesive(s) and as or in (a) coating composition(s) for the textile, paper, printing and leather industry.

The examples below are intended to illustrate the invention by way of example without limiting it in any way.

### EXAMPLES

The polymers can be prepared in customary ways known to the person skilled in the art. Preference is given to solution polymerization in alcohol and/or water, and particular preference to emulsion polymerization.

### Abbreviations:

MM Methacrylic acid
AA Acrylic acid
ITA Itaconic acid
UMA Ureido methacrylate
MMA Methyl methacrylate
EMA Ethyl methacrylate
n-BMA n-Butyl methacrylate
t-BMA t-Butyl methacrylate
t-BA t-Butyl acrylate
EA Ethyl acrylate
EHA Ethylhexyl acrylate
n-BA n-Butyl acrylate
HEMA Hydroxyethyl methacrylate
HEA Hydroxyethyl acrylate
HPA Hydroxypropyl acrylate
w/w in the weight ratio
AI Active ingredient
DME Dimethyl ether

### Preparation of the Polymers by Emulsion Polymerization

### Example 1

### Ethyl methacrylate/methacrylic acid 75/25 w/w

In a 2 l polymerization vessel with stirring and heating and cooling devices, at a temperature of from 20 to 25° C.,

| | | |
|---|---|---|
| 250 | g | of deionized water |
| 0.6 | g | of a 15% strength by weight aqueous solution of |
| | | sodium lauryl sulfate in deionized water |
| 35 | g | of feed II (see below) |

were initially introduced and heated to 45° C. with stirring and under a nitrogen atmosphere. After reaching the temperature, feed I (see below) was added over the course of 5 minutes.

The mixture was then heated to 80° C. and, while stirring and maintaining the reaction temperature, the remaining feed 11 was metered in over the course of 2.5 hours with constant feed streams.

When the feeds were complete, the reaction mixture was stirred for a further hour at 80° C. and then cooled to 60°.

While maintaining the temperature of 60°, feed III (see below) was added.

The mixture was then cooled to 35° C. and, while maintaining the reaction temperature, feed IV (see below) was added.

Feed I:

5 g of 7% strength by weight aqueous solution of sodium persulfate in deionized water

Feed II is an aqueous monomer emulsion prepared from:

| Initial | | % by wt. based |
|---|---|---|
| weight | | on the total |
| [g] | | amount of monomer |
| 120 | deionized water | |
| 5 | a 15% strength by weight aqueous solution | |
| | of sodium lauryl sulfate in deionized water | |
| 10 | nonionic emulsifier* | |
| 182 | ethyl methacrylate | 75 |
| 61 | methacrylic acid | 25 |
| 1.8 | n-dodecylmercaptan | |

*Tween ™ 80 can, for example, be used as nonionic emulsifier.

### Preparation of Feed II

To the initial charge of deionized water are added, with stirring, the total amount of the 15% strength by weight aqueous solution of sodium lauryl sulfate and then the total amount of the nonionic emulsifier. The appropriate amounts of ethyl methacrylate, methacrylic acid and n-dodecylmercaptan are added in the order given to the homogeneous solution, which continues to be stirred.

### Feed III:

2 g of 30% strength by weight solution of hydrogen peroxide in deionized water

### Feed IV:

40 g of 10% strength by weight solution of ammonium hydrogencarbonate in deionized water

The polymers of Examples 2 to 20 were synthesized analogously to Example 1, feed 11 for each example being chosen accordingly as stated below. Emulsifiers/water/batch size/emulsion preparation/regulator analogous to Example 1.

### Example 2

### Ethyl methacrylate/t-butyl acrylate/methacrylic acid 50/25/25 w/w/w

| | % by wt. based on the |
|---|---|
| | total mass of the monomers |
| Ethyl methacrylate | 50 |
| t-Butyl acrylate | 25 |
| Methacrylic acid | 25 |

### Example 3

### n-Butyl methacrylate/methacrylic acid 75/25 w/w (comparative example)

| | % by wt. based on the |
|---|---|
| | total mass of the monomers |
| n-Butyl methacrylate | 75 |
| Methacrylic acid | 25 |

### Example 4

### t-Butyl methacrylate/methacrylic acid 75/25 w/w (comparative example)

| | % by wt. based on the |
|---|---|
| | total mass of the monomers |
| t-Butyl methacrylate | 75 |
| Methacrylic acid | 25 |

### Example 5

### Methyl methacrylate/methacrylic Acid 75/25 w/w

### Comparative Example

| | % by wt. based on the |
|---|---|
| | total mass of the monomers |
| Methyl methacrylate | 75 |
| Methacrylic acid | 25 |

The polymers of Examples 6 to 20 below were synthesized analogously to Example 1, feed II for each example being chosen accordingly as stated below. Emulsifiers/water/batch size/emulsion preparation/regulator analogous to Example 1.

### Example 21

### Ethyl methacrylate/acrylic acid/methacrylic acid 75/5/20 w/w/w

In a 2 l polymerization vessel with stirrer and heating and cooling devices, at a temperature of from 20 to 25° C.,

| | | |
|---|---|---|
| 250 | g | of deionized water |
| 0.6 | g | of a 15% strength by weight aqueous solution of |
| | | sodium lauryl sulfate in deionized water |
| 35 | g | of feed II (see below) |

were initially introduced and heated to 45° C. with stirring and under a nitrogen atmosphere. After reaching the temperature, feed I (see below) was added over the course of 5 minutes. The mixture was then heated to 80° C. and, while stirring and maintaining the reaction temperature, the remaining feed II was metered in over the course of 2.5 hours with constant feed streams.

When the feeds were complete, the reaction mixture was stirred for a further hour at 80° C. and then cooled to 60°.

While maintaining the temperature of 60°, feed III (see below) was added.

The mixture was then cooled to 35° C. and, while maintaining the reaction temperature, feed IV (see below) was added.

### Feed 1:

5 g of 7% strength by weight aqueous solution of sodium persulfate in deionized water

Feed II is an aqueous monomer emulsion prepared from:

| Initial | | % by wt. based |
|---|---|---|
| weight | | on the total |
| [g] | | amount of monomer |
| 120 | deionized water | |
| 5 | a 15% strength by weight aqueous | |
| | solution of sodium lauryl sulfate in | |
| | deionized water | |
| 10 | nonionic emulsifier* | |
| 182 | ethyl methacrylate | 75 |
| 49 | methacrylic acid | 20 |
| 12 | acrylic acid | 5 |
| 2.1 | n-dodecyl mercaptan | |

*TweenTM 80 can, for example, be used as nonionic emulsifier.

### Feed II

To the initial charge of deionized water are added, with stirring, the total amount of the 15% strength by weight aqueous solution of sodium lauryl sulfate and then the total amount of the nonionic emulsifier. The appropriate amounts of ethyl methacrylate, methacrylic acid and n-dodecylmercaptan are added in the order given to the homogeneous solution, which continues to be stirred.

### Feed III:

2 g of 30% strength by weight solution of hydrogen peroxide in deionized water

### Feed IV:

40 g of 10% strength by weight solution of ammonium hydrogencarbonate in deionized water

The polymers of Examples 22 to 25 were synthesized analogously to Example 21, feed II for each example being chosen accordingly as stated below. Emulsifiers/water/batch size/emulsion preparation/regulator analogous to Example 21.

| Ex. | MAA | AA | ITA | EMA | UMA | EA | n-BA | HEA | HPA | EHA | HEMA | t-BA |
|---|---|---|---|---|---|---|---|---|---|---|---|---|
| 6 | 20 | | 5 | 75 | | | | | | | | |
| 7 | 40 | | | 60 | | | | | | | | |
| 8 | 25 | | | 60 | 15 | | | | | | | |
| 9 | 25 | | | 60 | | 15 | | | | | | |
| 10 | 25 | | | 60 | | | 15 | | | | | |
| 11 | 25 | | | 60 | | | | 15 | | | | |
| 12 | 25 | | | 60 | | | | | 15 | | | |
| 13 | 25 | | | 60 | | | | | | 15 | | |
| 14 | 32 | | | 43 | | | | | | 25 | | |
| 15 | 16 | | | 44 | | | | | | | | 40 |
| 16 | 16 | | | 24 | | | | | | | | 60 |
| 17 | 16 | | | 60 | | | | | | | 24 | |
| 18 | 16 | | | 64 | | | | | | | | 20 |
| 19 | 40 | | | 25 | | | | | | | 25 | 10 |
| 20 | 16 | | | 84 | | | | | | | | |
| 21 | 20 | 5 | | 75 | | | | | | | | |
| 22 | 15 | 10 | | 75 | | | | | | | | |
| 23 | 20 | 5 | | 75 | | | | | | | | |
| 24 | 15 | 5 | | 80 | | | | | | | | |
| 25 | 10 | 10 | | 80 | | | | | | | | |

### Measurement Methods

### 1) Determination of the K Value

The K values are measured in accordance with Fikentscher, Cellulosechemie, Vol. 13, pp. 58 to 64 (1932) at 25° C. in aqueous/ethanolic or ethanolic solution and are a measure of the molar weight. The aqueous/ethanolic or ethanolic solution of the polymers comprises 1 g of polymer in 100 ml of solution. In cases where the polymers are present in the form of aqueous dispersions, appropriate amounts of the dispersion are made up to 100 ml with ethanol depending on the polymer content of the dispersion so that the concentration is 1 g of polymer in 100 ml of solution.

The K value is measured in a Micro-Ubbelohde capillary model M Ic from Schott.

Determination of the Particle Size Distribution (PSD) using Malvern® Scattered Light Analysis

The particle size distribution was determined using a particle size measurement system for analyzing liquid aerosols “Malvern®Master Sizer X”.

(Malvern Instruments Inc., Southborough Mass., USA).

### Measurement Principle:

The measurement system is based on the method of laser light diffraction at the particle which, apart from spray analysis (aerosols, pump sprays), is also suitable for determining the size of solids, suspensions and emulsions in the size range from 0.1 μm to 2000 μm.

A particle collective (=droplet) is illuminated by a laser. On each droplet, some of the incident laser light is scattered. This light is received on a multielement detector and the associated photo energy distribution is determined. From this data, the associated particle distribution is calculated using the evaluation software.

### Procedure:

The aerosols were sprayed in at a distance of 29.5 cm to the laser beam. The spray cone entered at a right angle to the laser beam.

Before each measurement, the aerosol cans were fixed to a firmly installed holding device to ensure that all of the aerosols to be tested were measured at exactly the same distance.

Before the actual particle measurement, a “background measurement” was carried out. This eliminated the effects of dust and other contaminants in the measurement area.

The aerosol was then sprayed into the test space. The total particle volume was ascertained over a test period of 2 s and evaluated.

### Evaluation:

The evaluation comprises a tabular representation over 32 class widths from 0.5 μm to 2000 μm and additionally a graphical representation of the particle size distribution.

Since the spray experiments are an approximately uniform distribution, the mean diameter D (v, 0.5) is given. This numerical value indicates that 50% of the particle volume measured in total is below this value.

For readily sprayable aerosol systems in the cosmetics sector this value is between 30 μm to 80 μm depending on polymer content, valve, spray head geometry, solvent ratio and amounts of propellant gas.

### Setting (Flexural Rigidity).

The setting of polymeric film formers was measured not only by subjective assessment, but also in an objectively physical manner as the flexural rigidity of thin tresses of hair which had been treated with the polymer solution and dried again. In this method, a force transducer determines the force required for the bending, while the entire measurement is carried out under standardized conditions in a climatically controlled room at 65% relative atmospheric humidity.

To measure the flexural rigidity, 3.0% strength by weight solutions of the polymers according to the invention were prepared. The flexural rigidity was measured on 5 to 10 hair tresses (each about 3 g and 24 cm in length) at 20° C. and 65% relative humidity. The weighed dry hair tresses were immersed into the 3.0% strength by weight polymer solution, with triple immersion and removal ensuring uniform distribution. The excess film former solution was then stripped off between thumb and forefinger and the hair tresses were then carefully pressed by squeezing between filter paper. The tresses were then shaped by hand such that they had a round cross section.

Drying was carried out overnight at 20° C. and 65% relative humidity in the climatically controlled room.

The tests were carried out in a climatically controlled room at 20° C. and 65% relative humidity using a tensile/pressure testing instrument. The hair tress was placed symmetrically on two cylindrical rolls of the sample holder. The tress was then bent exactly in the middle from above using a 40 mm rounded punch (breakage of the polymer film). The force required for this was measured using a load cell (50 N) and given in Newtons. The values determined in this way were compared with those of a standard commercial comparative polymer (Amphomer® LV 71 as standard 100%, as stated).

### Ability to be Washed Out:

The hair tress was washed in an approximately 37° C.-hot Texapon®NSO solution (6 ml of Texapon®NSO (28% strength) in 1 l of warm water) for about 15 seconds by immersion and squeezing 5 times. The hair tress was then rinsed clear and treated again in the same way. The hair tress was then squeezed thoroughly on filter paper and left to dry overnight. The dry hair tress was rolled and investigated for residues.

Performance Properties of Some of the Polymers of Examples 1 to 20:

| | | | | | | PSD-Malvern, |
|---|---|---|---|---|---|---|
| | | | | | | 5% Al***, |
| | | | | | Ability to be | VOC 55 with |
| | | Weight ratio | | | washed out | 40% DME, [μm] |
| | | of monomers | K | Setting | in surfactant/ | VOC 55 |
| Ex. | Composition | (w/w/w) | value | [%] | water** | standard valve⁴* |
| 1 | EMA/MAA | 75/25 | 31 | 115 | good | 60 |
| 2 | t-BA/MAA/EMA | 25/25/50 | 32 | 100 | good | 70 |
| 3* | n-BMA/MAA | 75/25 | 30 | 70 | good | 70 |
| 4* | t-BA/MAA | 75/25 | 32 | 70 | good | 70 |
| 5* | MMA/MAA | 75/25 | 26 | 90 | poor | 70 |
| 6 | EMA/ITA/MAA | 75/5/20 | 30 | 110 | good | 60 |
| 7⁶* | EMA/MAA | 60/40 | 35 | 115 | good | 70 |
| 14 | EA/HEMA/MAA | 43/25/32 | 40 | 100 | good | 70 |
| 15 | t-BA/EMA/MAA | 40/44/16 | 37 | 100 | good | 65 |
| 16 | t-BA/EMA/MAA | 60/24/16 | 38 | 85 | good | 85 |
| 17 | EMA/HEMA/MAA | 60/24/16 | 37 | 110 | still good | 55 |
| 18 | t-BA/EMA/MAA | 20/65/15 | 36 | 85 | good | 80 |
| 20 | EMA/MAA | 84/16 | 34 | 125 | still good | 60 |
| 21 | EMA/AS/MAS | 75/5/20 | 31 | 110 | good | 70 |
| 22 | EMA/AS/MAS | 75/10/15 | 33 | 90 | good | 70 |
| 23 | EMA/AS/MAS | 75/5/20 | 31 | 110 | good | 60 |
| 24 | EMA/AS/MAS | 80/5/15 | 29 | 100 | good | 60 |
| 25 | EMA/AS/MAS | 80/10/10 | 30 | — | good | 55 |
| | Amphomer ® LV 71 | | | 100 | good | 160 |

*comparative experiments
**the surfactant used was Texapon NSO (Cognis)
***5% Al means that the characterized preparation comprised 5% by weight of polymer
⁴*the standard valve used was the valve Seaquist Perfect 1 Ariane M (Seaquist Perfect Dispensing GmbH)
⁵*sample was 60% neutralized, all of the other samples were 100% neutralized.

1-19. (canceled)

20. A copolymer prepared by a process comprising free-radical polymerizing:
(a) more than 15 to 40% by weight of at least one monoethylenically unsaturated carboxylic acid;
(b) 30 to less than 85% by weight of ethyl methacrylate; and
(c) 0 to 50% by weight of at least one additional free-radically polymerizable monomer other than the at least one monoethylenically unsaturated carboxylic acid and ethyl methacrylate;
wherein a total amount of the at least one monoethylenically unsaturated carboxylic acid, ethyl methacrylate and at least one additional free-radically polymerizable monomer equals 100% by weight;
with the proviso that where the process comprises a solution polymerization, the at least one monoethylenically unsaturated carboxylic acid consists of acrylic acid and methacrylic acid, and the at least one additional free-radically polymerizable monomer is an ester of (meth)acrylic acid and an aliphatic C₈-C₁₈-alcohol, then the ester of (meth)acrylic acid and an aliphatic C₈-C₁₈-alcohol is not present in an amount of 5 to 40% by weight; and
with the proviso that where the at least one additional free-radically polymerizable monomer is selected from the group consisting of N-vinylpyrrolidone, N-vinylcaprolactam and mixtures thereof, then the N-vinylpyrrolidone, N-vinylcaprolactam or mixture thereof is present in an amount of less than 5% by weight.

21. The copolymer according to claim 20, wherein the at least one monoethylenically unsaturated carboxylic acid is present in an amount of 18 to 35% by weight and the ethyl methacrylate is present in an amount of 40 to 82% by weight.

22. The copolymer according to claim 20, wherein the at least one monoethylenically unsaturated carboxylic acid is present in an amount of 20 to 35% by weight and the ethyl methacrylate is present in an amount of 45 to 80% by weight.

23. The copolymer according to claim 20, wherein the at least one monoethylenically unsaturated carboxylic acid consists of methacrylic acid and upto one or more additional components selected from the group consisting of acrylic acid, ethacrylic acid, crotonic acid, maleic acid, maleic anhydride, fumaric acid, itaconic acid, citraconic acid, mesaconic acid, glutaconic acid, aconitic acid and mixtures thereof.

24. The copolymer according to claim 20, wherein the at least one monoethylenically unsaturated carboxylic acid consists of methacrylic acid and upto one or more additional monoethylenically unsaturated monocarboxylic acids.

25. The copolymer according to claim 23, wherein the methacrylic acid and one or more additional components are present in a weight ratio greater than 2:1.

26. The copolymer according to claim 24, wherein the methacrylic acid and one or more additional monoethylenically unsaturated monocarboxylic acids are present in a weight ratio greater than 2:1.

27. The copolymer according to claim 20, wherein the at least one additional free-radically polymerizable monomer comprises a compound selected from the group consisting of acrylamide, methacrylamide, C₁-C₁₈-alkyl acrylates, C₁-C₁₈-alkyl methacrylates, N—C₁-C₁₈-alkylacrylamides and N—C₁-C₁₈-alkylmethacrylamides.

28. The copolymer according to claim 20, wherein the at least one additional free-radically polymerizable monomer comprises a compound selected from the group consisting of n-butyl acrylate, tert-butyl acrylate, hydroxyethyl methacrylate, ethyl acrylate and mixtures thereof.

29. The copolymer according to claim 20, wherein the at least one additional free-radically polymerizable monomer comprises tert-butyl acrylate.

30. A cosmetic preparation comprising a copolymer according to claim 20 and a cosmetically acceptable carrier.

31. The cosmetic preparation according to claim 30, wherein the cosmetically acceptable carrier comprises a component selected from the group consisting of: water; water-miscible organic solvents; oils, fats, waxes and esters of C₆-C₃₀-monocarboxylic acids with mono-, di- or trihydric alcohols; saturated acyclic and cyclic hydrocarbons; fatty acids; fatty alcohols; propellant gases; and mixtures thereof.

32. The cosmetic preparation according to claim 30, wherein the cosmetically acceptable carrier comprises a C₂₋₄ alkanol.

33. The cosmetic preparation according to claim 30, wherein the cosmetically acceptable carrier comprises ethanol.

34. A method comprising:
(a) providing a composition comprising a copolymer according to claim 20; and
(b) bringing the composition into contact with hair.

35. The method according to claim 34, further comprising a hair treatment selected from one or more of washing, coloring, bleaching, permanent waving and straightening.

36. The method according to claim 35, wherein the composition is brought into contact with the hair before the hair treatment.

37. The method according to claim 35, wherein the composition is brought into contact with the hair after the hair treatment.

38. The method according to claim 35, further comprising rinsing the hair after contact with the composition.
